(19) United States
(12) Patent Application Publication
(10) Pub. No.: US 2003/0073812 A1
(43) Pub. Date: Apr. 17, 2003

(54) Non-endogenous, constitutively activated versions of plant G protein-coupled receptor: GCR1

(76) Inventor: Gabriella Colucci, La Jolla, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/164,163

(22) Filed: Jun. 5, 2002

Related U.S. Application Data
(60) Provisional application No. 60/372,131, filed on Apr. 12, 2002.
(60) Provisional application No. 60/339,281, filed on Dec. 11, 2001.
(60) Provisional application No. 60/330,363, filed on Oct. 18, 2001.
(60) Provisional application No. 60/308,267, filed on Jul. 26, 2001.
(60) Provisional application No. 60/295,948, filed on Jun. 5, 2001.

(51) Int. Cl. A01N 25/00; C07K 14/415; C07H 21/04; C12P 21/02; C12N 5/04

(52) U.S. Cl. 530/350; 504/116.1; 536/23.6; 435/69.1; 435/320.1; 435/419

(57) ABSTRACT

The invention relates to transmembrane receptors for which the endogenous ligand has not been identified, and specifically to a plant GPCR (“GCR1”) that has been altered to establish constitutive activity of the receptor. In some embodiments, the altered versions of GCR1 are used for the direct identification of candidate compounds as receptor agonists, inverse agonists or partial agonists for use in, for example and not limitation, herbicidal relevance; germination; growth elongation; seed dormancy; and fruit and vegetable ripening and development. In some embodiments, altered versions of GCR1 are used to modulate physiological processes in a plant. The invention further relates to plants comprising constitutively activated non-endogenous GPCRs.

### CROSS-REFERENCE TO RELATED APPLICATIONS

This patent application claims priority benefit of U.S. Provisional Application No. 60/372,131, filed on Apr. 12, 2002; U.S. Provisional Application No. 60/339,281, filed on Dec. 11, 2001; U.S. Provisional Application No. 60/330,363, filed on Oct. 18, 2001; U.S. Provisional Application No. 60/308,267, filed on Jul. 26, 2001; and U.S. Provisional Application No. 60/295,948, filed on Jun. 5, 2001. Each of the foregoing applications is incorporated by reference in its entirety.

### FIELD OF THE INVENTION

The invention disclosed in this patent document relates to transmembrane receptors, and more particularly to a G protein-coupled receptor (“GPCR”) for which the endogenous ligand has not been identified; and specifically to a plant GPCR (“GCR1”) that has been altered to establish constitutive activity of the receptor. In some embodiments the altered versions of GCR1 are used, inter alia, for the direct identification of candidate compounds as receptor agonists, inverse agonists or partial agonists for use in, for example and not limitation, herbicidal relevance; germination; growth elongation; seed dormancy; drought tolerance; pesticide sensitivity; quiescence; cell-cycle regulation; flower development; and fruit and vegetable ripening and development.

### BACKGROUND OF THE INVENTION

GPCRs constitute a large number and functionally diverse superfamily of integral membrane proteins. These proteins are involved in the transduction of signals across cell membranes through the use of G proteins found intracellularly. Although GPCRs play a major role in this signaling pathway, to date there has only been one GPCR found in plants.

The wide variety of extracellular signaling molecules is mediated through GPCRs. Despite the array of agonists that bind to and stimulate GPCRs, these receptors share a common structural motif, having seven sequences of between 22 to 24 hydrophobic amino acids that form seven alpha helices, each of which spans the membrane (each span is identified by number, i.e., transmembrane-1 (TM-I), transmebrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz and Go are G proteins that have been identified. Endogenous ligand-activated GPCR coupling with the G-protein begins a signaling cascade process (referred to as “signal transduction). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and “active” state. A receptor in an inactive state is unable to link to the intracellular signaling transduction pathway to produce a biological response. Changing the receptor conformation to the active state allows linkage to the transduction pathway (via the G-protein) and produces a biological response.

A receptor may be stabilized in an active state by an endogenous ligand or a compound such as a drug. Recent discoveries, including but not exclusively limited to modifications to the amino acid sequence of the receptor, provide means other than endogenous ligands or drugs to promote and stabilize the receptor in the active state conformation. These means effectively stabilize the receptor in an active state by simulating the effect of an endogenous ligand binding to the receptor. Stabilization by such ligand-independent means is termed “constitutive receptor activation.”

GPCRs have been identified in vertebrates, invertebrates, arthropods, insects nematodes, fungi, yeast, and viruses, but have only recently been discovered in plants. It has been reported that the first plant GPCR has been cloned and characterized from Arabidopsis thaliana. (See, Josefsson, L. et al., Eur. J. Biochem. 249:415-450 (1997)).

### SUMMARY OF THE INVENTION

Disclosed herein are non-endogenous versions of an endogenous, plant G protein coupled receptor, referred to as “GCR1”, and uses thereof.

In some aspects the present invention is directed to non-endogenous, constitutively activated plant G protein-coupled receptors (GPCR). In some embodiments, the plant GPCR has an amino acid sequence selected from the group consisting of SEQ ID NOS:10 and 12. In some embodiments, the plant GPCR is encoded by a nucleotide sequence selected from the group consisting of SEQ ID NOS: 9 and 11.

In further aspects the present invention is directed to plasmids comprising a vector and a cDNA selected from the group consisting of SEQ.ID.NOS: 9 and 11.

In some aspects the present invention is directed to host cells comprising a plasmid wherein the plasmid comprises a vector and a cDNA selected from the group consisting of SEQ.ID.NOS.:9 and 11.

In additional aspects the present invention is directed to methods for directly identifying a non-endogenous candidate compound as an agonist or an inverse agonist to an endogenous plant GPCR. The methods comprise the steps of: (a) subjecting the endogenous plant GPCR to constitutive receptor activation to create a non-endogenous, constitutively activated plant GPCR; (b) contacting the non-endogenous candidate compound with the non-endogenous, constitutively activated plant GPCR; and (c) identifying the non-endogenous candidate compound as an inverse agonist or an agonist to the constitutively activated plant GPCR by measuring at least a 20% difference in an intracellular signal induced by the contacted compound as compared with an intracellular signal in the absence of the contacted compound. In some embodiments the non-endogenous candidate compound is an agonist.

In some aspects the present invention is directed to methods for directly identifying a non-endogenous candidate compound as an agonist or an inverse agonist to an endogenous constitutively activated plant GPCR. The methods comprise the steps of: (a) contacting the non-endogenous candidate compound with the endogenous constitutively activated plant GPCR; and (b) identifying the non-endogenous candidate compound as an inverse agonist or an agonist to the endogenous constitutively activated plant GPCR by measuring at least a 20% difference in an intracellular signal induced by the contacted compound as compared with an intracellular signal in the absence of the contacted compound. In some embodiments, an endogenous ligand for the endogenous plant GPCR has not been identified. In some embodiments the non-endogenous candidate compound is an agonist.

In additional aspects the present invention is directed to compounds identified by the methods set forth above and described below.

In additional aspects the present invention is directed to compositions, including pharmaceutical compositions, comprising compounds directly identified by the methods of the present invention.

In some aspects the present invention is directed to methods of modulating a physiological process in a plant comprising subjecting an endogenous plant GPCR to constitutive receptor activation to create a non-endogenous, constitutively activated plant GPCR. The physiological process is thereby modulated. In some embodiments, the endogenous plant GPCR has an amino acid sequence selected from the group consisting of SEQ ID NOS: 10 and 12. In some embodiments the plant is selected from the group consisting Antophyta. In some embodiments the plant is selected from the group consisting of roses, daffodils, tobacco, carnation, freesia, cotton, geranium, iris, chrysanthemum, lily, orchid, sunflower, and tulips. In some embodiments the physiological process is selected from the group consisting of herbicide sensitivity, germination, growth elongation, seed dormancy, drought tolerance, pesticide sensitivity, quiescence, cell-cycle regulation, flower development, and fruit and vegetable ripening and development. In some embodiments the non-endogenous candidate compound is an agonist.

In additional aspects, the present invention is directed to methods of modulating a physiological process in a plant comprising: (a) subjecting an endogenous plant GPCR to constitutive receptor activation to create a non-endogenous, constitutively activated plant GPCR; and (b) contacting the non-endogenous, constitutively activated plant GPCR with a non-endogenous agonist or inverse agonist of said GPCR. The physiological process is thereby modulated. In some embodiments, the endogenous plant GPCR has an amino acid sequence selected from the group consisting of SEQ ID NOS:10 and 12. In some embodiments the plant is selected from the group consisting of roses, daffodils, tobacco, carnation, freesia, cotton, geranium, iris, chrysanthemum, lily, orchid, sunflower, and tulips. In some embodiments the physiological process is selected from the group consisting of herbicide sensitivity, germination, growth elongation, seed dormancy, drought tolerance, pesticide sensitivity, quiescence, cell-cycle regulation, flower development, and fruit and vegetable ripening and development. In some embodiments the non-endogenous candidate compound is an agonist.

In some aspects the present invention is directed to methods for directly identifying a non-endogenous candidate compound as a compound having activity selected from the group consisting of inverse agonist activity and agonist activity, to an endogenous, constitutively active plant G protein coupled cell surface receptor (GPCR) comprising the steps of: (a) contacting a non-endogenous candidate compound with a GPCR Fusion Protein, the GPCR Fusion Protein comprising the endogenous, constitutively active plant GPCR and a G protein; and (b) identifying the non-endogenous candidate compound as an inverse agonist or an agonist to the endogenous constitutively activated plant GPCR by measuring at least a 20% difference in an intracellular signal induced by the contacted compound as compared with an intracellular signal in the absence of the contacted compound. In some embodiments the non-endogenous candidate compound is an agonist.

In additional aspects the present invention is directed to methods for directly identifying a non-endogenous candidate compound as a compound having activity selected from the group consisting of inverse agonist activity and agonist activity, to an endogenous, constitutively active plant G protein coupled cell surface receptor (GPCR) comprising the steps of: (a) contacting a non-endogenous candidate compound with a GPCR Fusion Protein, the GPCR Fusion Protein comprising the endogenous, constitutively active plant GPCR and a G protein; and (b) determining whether a receptor functionality is modulated, wherein a change in receptor functionality is indicative of the candidate compound being an agonist or inverse agonist of said endogenous, constitutively active plant GPCR. In some embodiments the endogenous ligand for said endogenous, constitutively active plant GPCR has not been identified. In some embodiments the non-endogenous candidate compound is an agonist.

In some aspects the present invention is directed to GPCR Fusion Protein constructs comprising a constitutively active plant G protein coupled receptor and a G protein. In some embodiments, the constitutively active plant G protein coupled receptor is non-endogenous. In some embodiments, the GPCR Fusion Protein construct comprises constitutively active plant G protein coupled receptor comprises an amino acid sequence selected from the group consisting of SEQ.ID.NOS.:10 and 12. In some embodiments, the said G protein is Gα.

In some aspects the present invention is directed to methods for modulating a physiological process in a plant. The methods comprise the steps of: (a) subjecting an endogenous plant GPCR to constitutive receptor activation to create a non-endogenous, constitutively activated plant GPCR; (b) contacting the non-endogenous candidate compound with the non-endogenous, constitutively activated plant GPCR; (c) identifying the non-endogenous candidate compound as an inverse agonist or an agonist to the non-endogenous constitutively activated plant GPCR by measuring at least a 20% difference in an intracellular signal induced by the contacted compound as compared with an intracellular signal in the absence of the contacted compound; and (d) contacting the plant with the inverse agonist or agonist; whereby the physiological process in the plant is modulated.

In further aspects the present invention is directed to methods for modulating a physiological process in a plant. The methods comprise the steps of: (a) subjecting an endogenous plant GPCR to constitutive receptor activation to create a non-endogenous, constitutively activated plant GPCR; (b) contacting the non-endogenous candidate compound with the non-endogenous, constitutively activated plant GPCR to yield a plant comprising a non-endogenous constitutively activated GPCR; (c) identifying the non-endogenous candidate compound as an inverse agonist or an agonist to the non-endogenous constitutively activated plant GPCR by measuring at least a 20% difference in an intracellular signal induced by the contacted compound as compared with an intracellular signal in the absence of the contacted compound; and (d) contacting the plant comprising a non-endogenous constitutively activated GPCR with the inverse agonist or agonist; whereby the physiological process in the plant is modulated.

In other aspects the present invention is directed to a plant comprising a non-endogenous, constitutively activated plant G protein-coupled receptor (GPCR). In some embodiments, the G protein-coupled receptor has an amino acid sequence selected from the group consisting of SEQ ID NOS:10 and 12. In some embodiments, the G protein-coupled receptor is encoded by a nucleotide sequence selected from the group consisting of SEQ ID NOS: 9 and 11.

In some aspects the present invention is directed to plants contacted with an inverse agonist or agonist identified by (a) subjecting the endogenous plant GPCR to constitutive receptor activation to create a non-endogenous, constitutively activated plant GPCR; (b) contacting the non-endogenous candidate compound with the non-endogenous, constitutively activated plant GPCR; and (c) identifying the non-endogenous candidate compound as an inverse agonist or an agonist to the constitutively activated plant GPCR by measuring at least a 20% difference in an intracellular signal induced by the contacted compound as compared with an intracellular signal in the absence of the contacted compound.

In some aspects the present invention is directed to plants comprising a non-endogenous, constitutively activated plant G protein-coupled receptor (GPCR).

### BRIEF DESCRIPTION OF THE DRAWINGS

FIG. 1 is a comparative analysis of endogenous, non-constitutively active plant GCR1 (“wt”) and non-endogenous, constitutively activated versions of plant GCR1 (“N216K” and “R217P”) in an SRE Reporter assay, where the control is expression vector (“CMV”).

FIG. 2 depicts a plate containing T1 progeny of plant GCR1. The plate is a comparative analysis of over-expressed, endogenous GCR1 seedlings (“over-GCR1”), non-endogenous, constitutively activated version of GCR1 (“R217P”) seedlings and seedlings without GCR1 (“control”).

FIG. 3 depicts pots of plants containing primary transformants of GCR1. The pots of plants are a comparative analysis of non-endogenous, constitutively activated version of GCR1 (“R217P”) and plants without GCR1 (“control”).

FIG. 4 depicts a plate containing buds of T3 Arabidopsis. FIG. 4 shows T3 progeny overexpressed GCR1 (“over-GCR1”) and non-endogenous version GCR1 (“R217P”) compared with the control Aequorin (a control which measures calcium).

FIG. 5 is representation of the fragment corresponding to the chosen leafy sequence when amplified in the Buds (“B1” and “B2”) and flowers (“F”) of both wild-type plants and R217P version of GCR1. This data evidences that R217P version of GCR1 is constitutively activated and results in early flowering of the Arabidopsis plant.

FIGS. 6A-C depict the expression of the genes: AtMYB65 (6A) and PP2A (6B) in wild-type GCR1 (“wt”) and overexpressed GCR1 (“over-GCR1”) on day 2 and day 7 after sowing. FIG. 6C is a representation of expression of “over-GCR1” (over expressed GCR1), AtMYB65 and the PPA2 catalytic subunit genes in wild-type and over-GCR1 transformants following two weeks after sowing and when a number of wild-type seeds had formed plantlets.

FIGS. 7A-C depict the expression of the genes: wild-type GCR1 (“GCR-wt”), overexpressed GCRI (“over-GCRI”), and non-endogenous, constitutively activated version of GCR1 (“R217P”) in 330 mM Sucrose (7A). FIG. 7B depicts a 96 well plate with GCR1 in the presence of 0.03M sucrose to identify small molecule that act as agonists. FIG. 7C depicts a 96 well plate with GCR1 in the presence of 0.33M sucrose to identify small molecules that act as inverse agonists.

FIG. 8 depicts an analysis of GCR1 mRNA abundance. Each lane contains 10 μg of RNA from six different BY-2 transformed lines (1 to 6). GCR1 coding sequence was used as a probe.

FIG. 9 depicts a GTPγ[³⁵S] binding assay. Receptor activity was measured in membrane preparations by determining the binding of the non-hydrolyzable GTP analog GTPγ[³⁵S]. Binding of GTPγ[³⁵S] in Arabidopsis T2 plants overexpressing GCR1 (hatched bar) was significantly higher compared to the wt Arabidopsis plant (solid bar). The experiment was performed three times and the results shown are representative. Error bars indicate S.D.

FIG. 10 is a graphic representation of the effect of the R271P version of GCR1 on the cell cycle. Control cell (empty vector), wt GCR1 (“wt”) and non-endogenous, constitutively activated version of GCR1 (R271P) overexpressing BY-2 cells were synchronized with aphidicolin for 24 h, and after removal of the cell cycle blocker thymidine incorporation was measured.

FIGS. 11A-B depict thymidine incorporation in the presence of ABA. FIG. 11A depicts wild-type GCR1 (“wt”) in the presence of 31M and 10 μM of ABA. FIG. 11B depicts a non-endogenous, constitutively activated version of GCR1 (“R271P”) in the presence of 3 μM and 10 μM of ABA.

### DETAILED DESCRIPTION

The scientific literature that has evolved around receptors has adopted a number of terms to refer to ligands having various effects on receptors. For clarity and consistency, the following definitions will be used throughout this patent document. To the extent that these definitions conflict with other definitions for these terms, the following definitions shall control.

AGONISTS shall mean materials (e.g., ligands, candidate compounds) that activate the intracellular response when they bind to the receptor, or enhance GTP binding to membranes.

AMINO ACID ABBREVIATIONS used herein are set out in Table A:

TABLE A

| | | |
|---|---|---|
| ALANINE | ALA | A |
| ARGININE | ARG | R |
| ASPARAGINE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

PARTIAL AGONISTS shall mean materials (e.g., ligands, candidate compounds) that activate the intracellular response when they bind to the receptor to a lesser degree/extent than do agonists, or enhance GTP binding to membranes to a lesser degree/extent than do agonists.

ANTAGONIST shall mean materials (e.g., ligands, candidate compounds) that competitively bind to the receptor at the same site as the agonists but which do not activate the intracellular response initiated by the active form of the receptor, and can thereby inhibit the intracellular responses by agonists or partial agonists. ANTAGONISTS do not diminish the baseline intracellular response in the absence of an agonist or partial agonist.

CANDIDATE COMPOUND, in the context of the disclosed invention, shall mean a small molecule (for example, and not limitation, a chemical compound) that is amenable to a screening technique.

CELL-CYCLE REGULATION, as used herein refers to the processes and mechanisms that are regulated during the division a parent cell into to two daughter cells. In some embodiments the cell cycle is regulated at the G1-S transition. In some other embodiments the cell cycle is regulated at the S-G2 transition. In other embodiments the cell cycle is regulated at the G2-M transition. And in some further embodiments the cell cycle is regulated when the cell exits mitosis and reenters G1 or enters into quiescence. In some other embodiments the cell cycle is regulated as a cell exits quiescence and enters into the process of cell division.

COMPOSITION means a material comprising at least one component; a “pharmaceutical composition” is an example of a composition.

COMPOUND EFFICACY shall mean a measurement of the ability of a compound to inhibit or stimulate receptor functionality, as opposed to receptor binding affinity. Exemplary means of detecting compound efficacy are disclosed in the Example section of this patent document.

CODON shall mean a grouping of three nucleotides (or equivalents to nucleotides) which generally comprise a nucleoside (adenosine (A), guanosine (G), cytidine (C), uridine (U) and thymidine (T)) coupled to a phosphate group and which, when translated, encodes an amino acid.

CONSTITUTIVELY ACTIVATED RECEPTOR shall mean a receptor subject to constitutive receptor activation. A constitutively activated receptor can be endogenous or non-endogenous.

CONSTITUTIVE RECEPTOR ACTIVATION shall mean stabilization of a receptor in the active state by means other than binding of the receptor with its endogenous ligand or a chemical equivalent thereof.

CONSTITUTIVELY ACTIVATING A GPCR IN A PLANT refers to the steps through which a non-endogenous plant GPCR is introduced into a plant.

CONTACT or CONTACTING shall mean bringing at least two moieties together, whether in an in vitro system or an in vivo system.

DIRECTLY IDENTIFYING or DIRECTLY IDENTIFIED, in relationship to the phrase “candidate compound”, shall mean the screening of an candidate compound against a constitutively activated receptor, preferably a constitutively activated receptor, and most preferably against a constitutively activated G protein-coupled cell surface receptor, and assessing the compound efficacy of such compound. This phrase is, under no circumstances, to be interpreted or understood to be encompassed by or to encompass the phrase “indirectly identifying” or “indirectly identified.”

DROUGHT TOLERANCE, as used herein refers to a plant being less or more susceptible to the lack of water. In some preferred embodiments a plant is less susceptible to the lack of water and its drought tolerance is therefore considered higher.

ENDOGENOUS shall mean a material that a mammal naturally produces. ENDOGENOUS in reference to, for example and not limitation, the term “receptor,” shall mean that which is naturally produced by a mammal (for example, and not limitation, a human) or a virus. By contrast, the term NON-ENDOGENOUS in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.”Both terms can be utilized to describe both “in vivo” and “in vitro” systems. For example, and not limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

The term EXPRESSION VECTOR shall refer to the molecules that comprise a nucleic acid sequence which encode one or more desired polypeptides and which include initiation and termination signals operably linked to regulatory elements including a promoter and polyadenylation signal capable of directing expression in the plant to be treated.

FLOWER DEVELOPMENT, as used herein, refers to the processes involved during the development of the floral meristem. In some embodiments the floral meristem develops into sepals, petals, stamen (male reproductive organs) and carpel (female reproductive organs).

FRUIT AND VEGETABLE RIPENING AND DEVELOPMENT shall refer to the processes by which a fruit or vegetable develops characteristic flavor, odor, body, texture, color, and the like.

GROWTH ELONGATION shall refer to the process of the roots and/or stem of a plant extending.

HERBICIDE SENSITIVITY shall refer to a plant being more or less susceptible to an herbicide. In some preferred embodiments the plant is less sensitive to herbicides.

HOST CELL shall mean a cell capable of having a Plasmid and/or Vector incorporated therein. In the case of a prokaryotic Host Cell, a Plasmid is typically replicated as a autonomous molecule as the Host Cell replicates (generally, the Plasmid is thereafter isolated for introduction into a eukaryotic Host Cell); in the case of a eukaryotic Host Cell, a Plasmid is integrated into the cellular DNA of the Host Cell such that when the eukaryotic Host Cell replicates, the Plasmid replicates. Preferably, for the purposes of the invention disclosed herein, the Host Cell is eukaryotic, more preferably, mammalian, and most preferably selected from the group consisting of 293, 293T and COS-7 cells.

INDIRECTLY IDENTIFYING or INDIRECTLY IDENTIFIED means the traditional approach to the drug discovery process involving identification of an endogenous ligand specific for an endogenous receptor, screening of candidate compounds against the receptor for determination of those which interfere and/or compete with the ligand-receptor interaction, and assessing the efficacy of the compound for affecting at least one second messenger pathway associated with the activated receptor.

INHIBIT or INHIBITING, in relationship to the term “response” shall mean that a response is decreased or prevented in the presence of a compound as opposed to in the absence of the compound.

INTRACELLULAR SIGNAL shall mean a detectable signal transduced by a receptor. Examples of intracellular signals are well-known to the art-skilled. Intracellular signals may be endogenous, e.g. an endogenous intracellular signal including without limitation second messengers; or non-endogenous, e.g. a non-endogenous intracellular signal including without limitation a engineered signal, i.e., β-galactosidase, GUS, luciferase. Assays for detecting intracellular signals are known to those skilled in the art and include GTPγS assays, cAMP assays; CREB assays; β-galactosidase assays; luciferase assays; DAG assays; AP1 assays; IP₃ assays; and adenylyl cyclase assays. In some embodiments the term INTRACELLULAR SIGNAL is used synonymously with “reporter signal”.

INTRODUCING shall mean methods of incorporating a compound into cells or tissues of a plant, and is used synonymously with “administering”.

INVERSE AGONISTS shall mean materials (e.g., ligand, candidate compounds) which bind to either the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

KNOWN RECEPTOR shall mean an endogenous receptor for which the endogenous ligand specific for that receptor has been identified.

LIGAND shall mean an endogenous, naturally occurring molecule specific for an endogenous, naturally occurring receptor.

MODULATE shall mean an increase or decrease in an amount, quality, or effect of a particular activity or protein.

MUTANT or MUTATION in reference to an endogenous receptor's nucleic acid and/or amino acid sequence shall mean a specified change or changes to such endogenous sequences such that a mutated form of an endogenous, non-constitutively activated receptor evidences constitutive activation of the receptor. In terms of equivalents to specific sequences, a subsequent mutated form of a human receptor is considered to be equivalent to a first mutation of the human receptor if (a) the level of constitutive activation of the subsequent mutated form of a human receptor is substantially the same as that evidenced by the first mutation of the receptor; and (b) the percent sequence (amino acid and/or nucleic acid) homology between the subsequent mutated form of the receptor and the first mutation of the receptor is at least about 80%, more preferably at least about 90% and most preferably at least 95%. Ideally, and owing to the fact that the most preferred cassettes disclosed herein for achieving constitutive activation includes a single amino acid and/or codon change between the endogenous and the non-endogenous forms of the GPCR, the percent sequence homology should be at least 98%.

NON-ORPHAN RECEPTOR shall mean an endogenous naturally occurring molecule specific for an endogenous naturally occurring ligand wherein the binding of a ligand to a receptor activates an intracellular signaling pathway.

ORPHAN RECEPTOR shall mean an endogenous receptor for which the endogenous ligand specific for that receptor has not been identified or is not known.

PESTICIDE SENSITIVITY, as used herein refers to the susceptibility of a plant or organism to pesticides. In a preferred embodiment the plant is less susceptible to pesticides.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

PLANT shall refer to all species of higher and lower plants of the Plant Kingdom. Plant embryos, seedlings, cells, cuttings, pieces, cells, tissues, tubers, rhizomes and seeds are included in the scope of the invention. In some embodiments the plant is a flowering ornamental, grain, vegetable, trees, trees for the production of lumber or paper, turf grasses, oil seed producing plants, plants that produce industrially relevant products, or fruit trees. In some embodiments the plant is selected from the group consisting Antophyta. Bioengineered plants are within the scope of the present invention. In some embodiments the plant is selected from the group consisting of roses, daffodils, tobacco, carnation, freesia, cotton, geranium, iris, chrysanthemum, lily, orchid, sunflower, rice, and tulips. Other plants are suitable for use in the present invention and include without limitation wheat, corn, soybean, barley, grain, tobacco, and rice.

PLASMID shall mean the combination of a Vector and cDNA. Generally, a Plasmid is introduced into a Host Cell for the purposes of replication and/or expression of the cDNA as a protein.

QUIESCENCE shall refer to the state of a cell that is capable of dividing but is currently not going through the cell cycle. This is normally termed G₀.

RECEPTOR FUNCTIONALITY shall refer to the normal operation of a receptor to receive a stimulus and moderate an effect in the cell, including, but not limited to regulating gene transcription, regulating the influx or efflux of ions, effecting a catalytic reaction, and/or modulating activity through G-proteins. RECEPTOR FUNCTIONALITY can readily be measured by the art skilled by measuring, without limitation, intracellular signals, ion influx or efflux, gene transcription, and effect of catalytic reaction

SECOND MESSENGER shall mean an intracellular response produced as a result of receptor activation. A second messenger can include, for example, inositol triphosphate (IP₃), diacycglycerol (DAG), cyclic AMP (cAMP), and cyclic GMP (cGMP). Second messenger response can be measured for a determination of receptor activation. In addition, second messenger response can be measured for the direct identification of candidate compounds, including for example, inverse agonists, agonists, partial agonists and antagonists.

SEED DORMANCY, as used herein refers to the state of a seed that is not growing, germinating, or otherwise developing.

SIGNAL TO NOISE RATIO shall mean the signal generated in response to activation, amplification, or stimulation wherein the signal is above the background noise or the basal level in response to non-activation, non-amplification, or non-stimulation. In some preferred embodiments, the signal is at least 10%, preferably at least 20%, more preferably at least 30%, more preferably at least 40%, more preferably at least 50%, more preferably at least 60%, more preferably at least 70%, more preferably at least 80%, more preferably at least 90%, and most preferably at least 100% above background noise or basal level.

SPACER shall mean a translated number of amino acids that are located after the last codon or last amino acid of a gene, for example a GPCR of interest, but before the start codon or beginning regions of the G protein of interest, wherein the translated number amino acids are placed in-frame with the beginnings regions of the G protein of interest. The number of translated amino acids can be tailored according to the needs of the skilled artisan and is generally from about one amino acid, preferably two amino acids, more preferably three amino acids, more preferably four amino acids, more preferably five amino acids, more preferably six amino acids, more preferably seven amino acids, more preferably eight amino acids, more preferably nine amino acids, more preferably ten amino acids, more preferably eleven amino acids, and even more preferably twelve amino acids.

STIMULATE or STIMULATING, in relationship to the term “response” shall mean that a response is increased in the presence of a compound as opposed to in the absence of the compound.

SUBJECTING AN ENDOGENOUS PLANT GPCR TO CONSTITUTIVE RECEPTOR ACTIVATION shall refer to the steps through which a plant GPCR is constitutively activated.

SUBSTANTIALLY SIMILAR shall refer to a result that is within 40% of a control result, preferably within 35%, more preferably within 30%, more preferably within 25%, more preferably within 20%, more preferably within 15%, more preferably within 10%, more preferably within 5%, more preferably within 2%, and most preferably within 1% of a control result. For example, in the context of receptor functionality, a test receptor may exhibit SUBSTANTIALLY SIMILAR results to a control receptor if the transduced signal, measured using a method taught herein or similar method known to the art-skilled, if within 40% of the signal produced by a control signal.

VECTOR in reference to cDNA shall mean a circular DNA capable of incorporating at least one cDNA and capable of incorporation into a Host Cell.

The order of the following sections is set forth for presentational efficiency and is not intended, nor should be construed, as a limitation on the disclosure or the claims to follow.

A. Introduction

Constitutively active forms of the plant G protein-coupled receptor GCR1, disclosed in the present patent document, can be obtained without limitation by site-directed mutational methods. Constitutively active receptors useful for direct identification of candidate compounds are most preferably achieved by mutating the receptor at a specific location, for example within transmembrane six (TM6) regions. Such mutations can produce a non-endogenous receptor that is constitutively activated, as evidenced by an increase in the functional activity of the receptor, for example, an increase in the level of second messenger activity.

The present invention relates to the plant GCR1. GCR1 was cloned and sequenced in 1997. (See, Josefsson, L. et al., Eur. J. Biochem. 249:415-450 (1997)). GCR1 is a GPCR having an open reading frame of 981 basepairs encoding a 326 amino acid protein.

In plants, several studies have implicated G protein, specifically, Gα subunits, in responses to hormones, light and pathogen resistance. (See, Hooley, R. 37 Plant Physiol. Biochem. 393 (1999)). No function has yet been reported for GCR1. (see, Plakidou-Dymock, S., et al. 8 Curr. Biol. 315 (1998)). However, it has been determined by measuring GTPγS binding that signaling through GCR1 is indeed coupled to Gα and that signaling through GCR1 is involved in at least two gibberellin (GA) dependent processes: seed dormancy and flowering. Overexpression of GCR1 in Arabidopsis abolished seed dormancy coincident with the upregulation of genes encoding the catalytic subunit of protein phosphatase PP2A and the transcription factor AtMYB65 in germinating seeds. Overexpression of GCR1 also produced an early flowering phenotype caused by the activation of the meristem identity gene LEAFY. Colucci et al. (Proc Natl Acad Sci USA (2002), 99(7): 4736-4741), incorporated herein by reference in its entirety, discusses a link between the overexpression of GCR1 and the abolition of seed dormancy and the shortened time to flowering.

As will be set forth and disclosed in greater detail below, utilization of several mutational approaches to modify the endogenous sequence of plant GCR1 leads to constitutively activated versions of this receptor. These non-endogenous, constitutively activated versions of plant GCR1 can be utilized, inter alia, for the screening of candidate compounds to directly identify compounds which modulate processes and activities including, but not limited to, herbicidal relevance; germination; growth elongation; seed dormancy; drought tolerance; pesticide sensitivity; quiescence; cell-cycle regulation; flower development; and fruit and vegetable ripening and development. Such physiological plant processes can further be modulated through, inter alia, subjecting an endogenous plant GPCR to constitutive receptor activation to create a non-endogenous, constitutively activated plant GPCR; and contacting the non-endogenous, constitutively activated plant GPCR with a non-endogenous agonist or inverse agonist of the GPCR, or, in other embodiments, by subjecting an endogenous plant GPCR to constitutive receptor activation to create a non-endogenous, constitutively activated plant GPCR, whereby the physiological process is modulated. The compositions and methods set forth herein are equally applicable to orphan and non-orphan GPCRs.

B. Receptor Screening

Screening candidate compounds against a non-endogenous, constitutively activated version of the plant GCR1, disclosed herein, allows for the direct identification of candidate compounds which act at the cell surface of the receptor, without requiring use of the receptor's endogenous ligand.

This patent document discloses several mutational approaches for creating non-endogenous, constitutively activated versions of plant GCR1. With the disclosed techniques, one skilled in the art is credited with the ability to create such constitutively activated versions of GCR1 for the uses disclosed herein, as well as other uses.

C. Screening of Candidate Compounds

1. Generic GPCR Screening Assay Techniques

When a G protein receptor becomes constitutively active, it binds to a G protein (e.g., Gq, Gs, Gi, Gz, Go) and stimulates the binding of GTP to the G protein. The G protein then acts as a GTPase and slowly hydrolyzes the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. An example of this monitoring, among other examples well-known and available to those in the art, was reported by Traynor and Nahorski in 1995. The preferred use of this assay system is for initial screening of candidate compounds because the system is generically applicable to all G protein-coupled receptors regardless of the particular G protein that interacts with the intracellular domain of the receptor.

2. Specific GPCR Screening Assay Techniques

Once candidate compounds are identified using the “generic” G protein-coupled receptor assay (i.e., an assay to select compounds that are agonists, partial agonists, or inverse agonists), further screening to confirm that the compounds have interacted at the receptor site is preferred. For example, a compound identified by the “generic” assay may not bind to the receptor, but may instead merely “uncouple” the G protein from the intracellular domain.

a. Gs, Gz and Gi.

Gs stimulates the enzyme adenylyl cyclase. Gi (and Gz and Go), on the other hand, inhibits this enzyme. Adenylyl cyclase catalyzes the conversion of ATP to cAMP; thus, constitutively activated GPCRs that couple the Gs protein are associated with increased cellular levels of cAMP. On the other hand, constitutively activated GPCRs that couple Gi (or Gz, Go) protein is associated with decreased cellular levels of cAMP. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Thus, assays that detect cAMP can be utilized to determine if a candidate compound is, e.g. an inverse agonist to the receptor (i.e., such a compound would decrease the levels of cAMP). A variety of approaches known in the art for measuring cAMP can be utilized; a most preferred approach relies upon the use of anti-cAMP antibodies in an ELISA-based format. Another type of assay that can be utilized is a second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, a constitutively activated Gs-linked receptor causes the accumulation of cAMP that then activates the gene and expression of the reporter protein. The reporter protein such as β-galactosidase or luciferase can then be detected using standard biochemical assays (Chen et al., Anal Biochem 1995;226(2):349-54).

b. Go and Gq.

Gq and Go are associated with activation of the enzyme phospholipase C, which in turn hydrolyzes the phospholipid PIP₂, releasing two intracellular messengers: diacycloglycerol (DAG) and inositol 1,4,5-triphoisphate (IP₃). Increased accumulation of IP₃ is associated with activation of Gq- and Go-associated receptors. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Assays that detect IP₃ accumulation can be utilized to determine if a candidate compound is, e.g., an inverse agonist to a Gq- or Go-associated receptor (i.e., such a compound would decrease the levels of IP₃). Gq-associated receptors can also be examined using an AP1 reporter assay in that Gq-dependent phospholipase C causes activation of genes containing AP1 elements; thus, activated Gq-associated receptors will evidence an increase in the expression of such genes, whereby inverse agonists thereto will evidence a decrease in such expression, and agonists will evidence an increase in such expression. Commercially available assays for such detection are available.

3. GPCR:G Protein Fusion Construct

The use of an endogenous, constitutively activate GPCR or a non-endogenous, constitutively activated GPCR, for use in screening of candidate compounds for the direct identification of inverse agonists, agonists and partial agonists provide an interesting screening challenge in that, by definition, the receptor is active even in the absence of an endogenous ligand bound thereto. Thus, in order to differentiate between, e.g., the non-endogenous receptor in the presence of a candidate compound and the non-endogenous receptor in the absence of that compound, with an aim of such a differentiation to allow for an understanding as to whether such compound may be an inverse agonist, agonist, partial agonist or have no affect on such a receptor, it is preferred that an approach be utilized that can enhance such differentiation. A preferred approach is the use of a GPCR Fusion Protein.

Generally, once it is determined that an endogenous or a non-endogenous GPCR has been constitutively activated (e.g., using the assay techniques set forth above, as well as others) it is possible to determine the predominant G protein that couples with the endogenous GPCR. Coupling of the G protein to the GPCR provides a signaling pathway that can be assessed because the non-endogenous, constitutively activated GPCR will continuously signal. In this regard, it is preferred that this signal be enhanced such that in the presence of, e.g., an inverse agonist to the receptor, it is more likely that it will be able to more readily differentiate, particularly in the context of screening, between the receptor when it is contacted with the inverse agonist.

The GPCR Fusion Protein is intended to enhance the efficacy of G protein coupling with the non-endogenous GPCR. The GPCR Fusion Protein is preferred for screening with a non-endogenous, constitutively activated GPCR because such an approach increases the signal that is most preferably utilized in such screening techniques. This is important in facilitating a significant “signal to noise” ratio; such a significant ratio is preferred for the screening of candidate compounds as disclosed herein.

The construction of a construct useful for expression of a GPCR Fusion Protein is within the purview of those having ordinary skill in the art. Commercially available expression vectors and systems offer a variety of approaches that can fit the particular needs of an investigator. The criteria of importance for such a GPCR Fusion Protein construct is that the endogenous GPCR sequence and the G protein sequence both be in-frame (preferably, the sequence for the endogenous GPCR is upstream of the G protein sequence) and that the “stop” codon of the GPCR must be deleted or replaced such that upon expression of the GPCR, the G protein can also be expressed. The GPCR can be linked directly to the G protein, or there can be spacer residues between the two (preferably, no more than about 12, although this number can be readily ascertained by one of ordinary skill in the art). Based upon convenience, use of a spacer is preferred in that some restriction sites that are not used will, effectively, upon expression, become a spacer. Most preferably, the G protein that couples to the non-endogenous GPCR will have been identified prior to the creation of the GPCR Fusion Protein construct. Because there are only a few G proteins that have been identified, it is preferred that a construct comprising the sequence of the G protein (i.e., a universal G protein construct) be available for insertion of an endogenous or a non-endogenous GPCR sequence therein; this provides for efficiency in the context of large-scale screening of a variety of different GPCRs having different sequences.

As noted above, constitutively activated GPCRs that couple to Gi, Gz and Go are expected to inhibit the formation of cAMP making assays based upon these types of GPCRs challenging (i.e., the cAMP signal decreases upon activation thus making the direct identification of, e.g, inverse agonists (which would further decrease this signal), interesting. As will be disclosed herein, it has been ascertained that for these types of receptors, it is possible to create a GPCR Fusion Protein that is not based upon the endogenous GPCR's endogenous G protein, in an effort to establish a viable cyclase-based assay. Thus, for example, an endogenous Gi coupled receptor can be fused to a Gs protein—it is believed that such a fusion construct, upon expression, “drives” or “forces” the endogenous GPCR to couple with, e.g., Gs rather than the “natural” Gi protein, such that a cyclase-based assay can be established. Thus, for Gi, Gz and Go coupled receptors, it is preferred that when a GPCR Fusion Protein is used and the assay is based upon detection of adenylyl cyclase activity, that the fusion construct be established with Gs (or an equivalent G protein that stimulates the formation of the enzyme adenylyl cyclase).

4. Co-Transfection of a Target Gi Coupled GPCR with a Fusion Protein Construct

A Gi coupled receptor is known to inhibit adenylyl cyclase, and, therefore, decrease the level of cAMP production, which can make assessment of cAMP levels challenging. An effective technique in measuring the decrease in production of cAMP as an indication of constitutive activation of a receptor that predominantly couples Gi upon activation can be accomplished by co-transfecting the Gi coupled receptor with a Fusion Protein Construct, wherein two different G proteins are fused together and co-transfected with a GPCR of interest. By way of example, and not limitation, a Gs protein can be fused with a Gi Protein, resulting in a “Gs/Gi Fusion Construct,” or a Gq protein fused with a Gs, Gi, Gz or Go Protein. With respect to Gq Fusion Protein constructs, a most preferred fusion construct can be accomplished wherein the first six (6) amino acids of the G-protein α-subunit (“Gαq”) is deleted and the last five (5) amino acids at the C-terminal end of Gαq is replaced with the corresponding amino acids of the Gα of the G protein of interest. For example, a fusion construct can have a Gq (6 amino acid deletion) fused with a Gi protein, resulting in a “Gq/Gi Fusion Construct”.

These fusion constructs are believed will force the endogenous Gi coupled receptor to couple to its non-endogenous G protein, for example Gs, such that the second messenger, for example, cAMP can be measured as an increase instead of a decrease, as is the case with Gi coupled receptors. As is apparent, constitutive activation of a Gs coupled receptor can be determined based upon an increase in production of cAMP. Constitutive activation of a Gi coupled receptor leads to a decrease in production cAMP. Thus, this co-transfection approach is intended to allow the ligand bound Gi coupled receptor to recognize and activate, for example, Gs protein, thereby stimulating the adenylyl cyclase activity and thus increase production of cAMP.

Screening of candidate compounds using a cAMP based assay can then be accomplished, with two provisos: first, relative to the Gi coupled target receptor, “opposite” effects will result, i.e., an inverse agonist of the Gi coupled target receptor will increase the measured cAMP signal, while an agonist of the Gi coupled target receptor will decrease this signal; second, as would be apparent, candidate compounds that are directly identified using this approach should be assessed independently to ensure that a differentiation in, for example, adenylyl cyclase, is not due to the expression of a fusion protein construct.

5. Co-Transfection of a Target Gi Coupled GPCR with a Signal-Enhancer Gs Coupled GPCR (cAMP Based Assays)

Another effective technique in measuring the decrease in production of cAMP as an indication of constitutive activation of a receptor that predominantly couples Gi upon activation can be accomplished by co-transfecting a signal enhancer, e.g., a non-endogenous, constitutively activated receptor that predominantly couples with Gs upon activation with the Gi linked GPCR. This co-transfection approach is intended to advantageously exploit these “opposite” affects. For example, co-transfection of a non-endogenous, constitutively activated Gs coupled receptor (the “signal enhancer”) with the endogenous Gi coupled receptor (the “target receptor”) provides a baseline cAMP signal (i.e., although the Gi coupled receptor will decrease cAMP levels, this “decrease” will be relative to the substantial increase in cAMP levels established by constitutively activated Gs coupled signal enhancer). By then co-transfecting the signal enhancer with a constitutively activated version of the target receptor, cAMP would be expected to further decrease (relative to base line) due to the increased functional activity of the Gi target (i.e., which decreases cAMP).

As above, screening of candidate compounds using a cAMP based assay can then be accomplished, with two provisos: first, relative to the Gi coupled target receptor, “opposite” effects will result, i.e., an inverse agonist of the Gi coupled target receptor will increase the measured cAMP signal, while an agonist of the Gi coupled target receptor will decrease this signal; second, as would be apparent, candidate compounds that are directly identified using this approach should be assessed independently to ensure that these do not target the signal enhancer receptor (this can be done prior to or after screening against the co-transfected receptors).

D. Preparation of Transgenic Plants

The art skilled are credited with the ability to introduce non-endogenous receptor sequences, including non-endogenous GPCR fusion protein sequences, into plants using, inter alia, the methods described below. According to some embodiments of the invention, non-endogenous receptor sequences are administered to the target plant by infection with Agrobacterium. In some embodiments, non-endogenous receptor sequences are introduced into a plant using a gene gun. Electroporation and microinjection may also be used to introduce non-endogenous receptor sequences into the target plant.

Administration to a plant may comprise transforming the plant with a construct comprising a promoter and a gene encoding the non-endogenous receptor or fusion protein thereof. An expression vector, comprising a promoter operably linked to at least one desired gene, may be used to achieve transformation of a plant. Initiation codons and stop codons are generally considered to be part of a nucleic acid sequence that encodes a polypeptide. However, it is necessary that these elements be functional in the cells of the plant to which the gene construct is administered, e.g., the initiation and termination codons are in frame with the coding sequence. Administration to the desired plant may be performed in conjunction with agents which facilitate the uptake and/or expression of the desired gene by the target plant. Such agents include without limitation polyethylene glycol, heat treatment of cells, and cold treatment of cells.

Desired genes within expression vectors administered to a plant are expressed under control of the gene promoter, producing the desired polypeptide. Typical expression vectors include, but are not limited to, the pB1 series of vectors and derivatives thereof, and green fluorescent protein reporter systems. Other methods of delivering sequences into plants are known to the art-skilled and include without limitation Ti-plasmid vectors, in vitro protoplast transformation, plant virus-mediated transformation, liposome-mediated transformation, gene gun, and ballistic particles. Additionally, U.S. Pat. No. 6,271,360 to Metz et al. discusses “Single-Stranded Oligodeoxynucleotide Mutational Vectors”. Such vectors may also be useful in transforming plant cells with, inter alia, constitutively active non-endogenous plant GPCRs.

Other examples of methods of introducing non-endogenous receptor sequences into plants are discussed in Hiei et al. (1994) Plant J 6: 271-282; Weigel et al., Arabidopsis, A Laboratory Manual, Chapter 5 entitled, How to Transform Arabidopsis, (2000), Cold Spring Harbor Laboratory Press, New York, 2002; and Melody S. Clark (Ed.), Plant Molecular Biology—A Laboratory Manual, Part III entitled, Genetic Engineering: Methodology and Analysis (1997), Springer-Verlag Publishers, Berlin, 1997; each of which is incorporated by reference in its entirety.

Transgenic plants comprising non-endogenous constitutively activated GPCRs can also be generated. The non-endogenous, constitutively activated GPCRs are generated by constitutively activating endogenous GPCRs. In some embodiments, endogenous GPCRs, for example GCR1, may be collected from the plants described herein, constitutively activated, and then reintroduced into the plant. In other embodiments, constitutively activated GPCRs, for example GCR1, from one plant species can be introduced into a different plant species, i.e. introducing a non-endogenous, constitutively active GPCR ortholog. Various permutations of the above are also contemplated. Reintroduction of modified nucleotide sequences into plants is known to those skilled in the art.

E. Other Utility

Although a preferred use of the non-endogenous versions of plant GPCRs disclosed herein may be for the direct identification of candidate compounds as inverse agonists, agonists or partial agonists, as discussed above, non-endogenous, constitutively activated plant GPCRs and GPCR Fusion constructs comprising same can be used to modulate physiological processes in plants. Additionally, these versions of the plant GPCR can also be utilized in research settings. For example, in vitro and in vivo systems incorporating GPCRs including GPCR Fusion Proteins can be utilized to further elucidate and better understand the roles these receptors play in the plant condition, as well as understanding the role of constitutive activation as it applies to understanding the signaling cascade. The value in non-endogenous versions of the plant GPCR is that their utility as a research tool is enhanced in that, because of their unique features, non-endogenous versions of the plant GPCR can be used to understand the role of these receptors in the plant before the endogenous ligand therefore is identified. Other uses of the disclosed receptors will become apparent to those in the art based upon, inter alia, a review of this patent document.

### EXAMPLES

The following examples are presented for purposes of elucidation, and not limitation, of the present invention. While specific nucleic acid and amino acid sequences are disclosed herein, those of ordinary skill in the art are credited with the ability to make minor modifications to these sequences while achieving the same or substantially similar results reported below (i.e., constitutive activation) disclosed herein. Such modified approaches are considered within the purview of this disclosure.

### Example 1

### Preparation of Endogenous GCR1

The full-length cDNA encoding GCR1 from Arabidopsis thaliana was kindly supplied by Dr. Richard Hooley, (IACR, University of Bristol, UK) cloned into EcoRI site of pCRII plasmid (Invitrogen). The pCRII Plasmid was excised using HindIII and XbaI and the released fragment was subsequently ligated into pcDNA3.1(+) (Invitrogen). Nucleic acid (SEQ.ID.NO.:1) and amino acid (SEQ.ID.NO.:2) sequences for plant GCR1 were thereafter determined and verified.

### Example 2

### Preparation of Non-Endogenous, Constitutively Activated Versions of Plant GCR1

Those skilled in the art are credited with the ability to select techniques for mutation of a nucleic acid sequence. Presented below are approaches utilized to create non-endogenous versions of the plant GCR1 disclosed above. The importance here is that the mutation leads to a constitutively activated receptor as disclosed herein, for the preferred uses disclosed herein.

For each mutation two primers (mutation underlined) that contained the same mutation and produced two products with an overlapping region were used. The overlapping primary products were subsequently re-amplified using the flanking primers (see, oligonucleotides 1 and 4 below) resulting in a full-length fragment containing the mutation. The primary for the primary reaction are listed in Table B below:

TABLE B

| GCR1 | | | |
|---|---|---|---|
| Mutation | Oligonucleotide | | |
| Identifier | No. | 5′-3′ orientation | |
| N216K | 1 | TCTAGACCCGGGATGTCGGCGGTTCTCACA | (sense; SEQ.ID.NO.:3) |
| | 2 | GAGTrAAAGGTG1ITGAAGAGATGGGGATACTATCCACTC | (sense; SEQ.ID.NO.:4) |
| | 3 | TGGATAGTATCCCCATCTCTTCAACACCTTTAACTC | (antisense; SEQ.ID.NO.:5) |
| | 4 | ATGATCTCGAGTCAACCCCTTTGCTGTCCACC | (antisense; SEQ.ID.NO.:6) |
| R217P | 1 | TCTAGACCCGGGATGTCGGCGGTTCTCACA | (sense; SEQ.ID.NO.:3) |
| | 5 | TTAAAGGTGTTGAACCCATGGGGATACTATCCACTC | (sense; SEQ.ID.NO.:7) |
| | 6 | ATAGTATCCCCATGGCTTCAACACCTTTAACTCTC | (anti-sense; SEQ.ID.NO.:8) |
| | 4 | ATGATCTCGAGTCAACCCTTTGCTGTCCACC | (antisense; SEQ.ID.NO.:6) |

The primary PCR products were then purified with the QIAquick PCR Purification kit (QIAGEN) used according to the manufacturer's instructions, and amplified together in a single second PRC reaction.

In the second PCR reaction, oligonucleotides 1 and 4 were used. Both, primary and secondary PCR reactions were performed in 100 μl containing 50 pmol of each primer, 0.4 mM DNA and 1.0 unit Pfu polymerase (Stratagene) in the corresponding buffer. The primary PCR reaction amplified the two separate fragments by 30 cycles, each cycle comprising 40 seconds at 94° C., 40 seconds at 48° C., 2 minute at 72° C. and an extension step at 72° C. for 10 minutes. The second PCR reaction amplified the recombinant fragment in 12 cycles using the same conditions disclosed above.

3′ A-overhangs were added to allow the cloning into a pCR2.1 Topo vector (Invitrogen), an additional 10 minute extension time was performed after adding 0.5 units of Ampli-Taq polymerase (Perkin Elmer).

The resulting 1,000 bp products were cloned in a pCR2.1 Topo vector, and sequenced by Sequenase™ dideoxy chain termination (United States Biochemical, according to manufacturer's instructions). The cloning plasmids were digested with XbaI and HindIII and ligated in the correspondent sites of the pcDNA3.1(+).

The non-endogenous versions of plant GCR1 were then sequenced. The derived and verified nucleic acid and amino acid sequences are listed in the accompanying “Sequence Listing” appended to this patent document, as summarized in Table C below:

TABLE C

| | Nucleic Acid Sequence | Amino Acid Sequence |
|---|---|---|
| Mutated GPCR | Listing | Listing |
| N216K | SEQ. ID. NO.: 9 | SEQ. ID. NO.: 10 |
| R217P | SEQ. ID. NO.: 11 | SEQ. ID. NO.: 12 |

Assessment of constitutive activity of the non-endogenous versions of plant GCR1 was then accomplished as disclosed below in Example 4.

### Example 3

### Receptor Expression

Although a variety of cells are available to the art for the expression of proteins, it is most preferred that mammalian cells be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells. Of the mammalian cells, COS-7, 293, 293T cells are particularly preferred, although the specific mammalian cell utilized can be predicated upon the particular needs of the artisan. The following approach was used for the indicated receptors, and can also be applied with respect to other receptors disclosed herein.

On day one, 3×10⁶/10 cm dish of 293 cells were plated out. On day two, two reaction tubes were prepared (the proportions to follow for each tube are per plate): tube A was prepared by mixing 4 μg DNA (e.g., pCMV vector; pCMV vector with receptor cDNA, etc.) in 0.5 ml serum free DMEM (Gibco BRL); tube B was prepared by mixing 24 μl Lipofectamine (Gibco BRL) in 0.5 ml serum free DMEM. Tubes A and B were admixed by inversions (several times), followed by incubation at room temperature for 30-45 min. The admixture is referred to as the “transfection mixture”. Plated 293 cells were washed with 1XPBS, followed by addition of 5 ml serum free DMEM. 1 ml of the transfection mixture were added to the cells, followed by incubation for 4 hrs at 37° C./5% CO₂. The transfection mixture was removed by aspiration, followed by the addition of 10 ml of DMEM/10% Fetal Bovine Serum. Cells were incubated at 37° C./5% CO₂. After 48 hr incubation, cells were harvested and utilized for analysis.

### Example 4

### Assays for Determination of Constitutive Activity of Non-Endogenous GCR1

A. SRE REPORTER ASSAY

A SRE-Luc Reporter (a component of Mercury Luciferase System 3, Clontech Catalogue # K2053-1) was utilized in 293 cells. Cells were transfected with the plasmid components of this system and the indicated expression plasmid encoding endogenous or non-endogenous receptor using Lipofectamine Reagent (Gibco/BRL, Catalogue #18324-012) according to the manufacturer's instructions. Briefly, 420 ng SRE-Luc, 50 ng CMV (comprising the receptor) and 30 ng CMV-SEAP (secreted alkaline phosphatase expression plasmid; alkaline phosphatase activity is measured in the media of transfected cells to control for variations in transfection efficiency between samples) were combined in a cationic lipid-DNA precipitate as per the manufacturer's instructions. The final volume was 25 μl brought up with Optimem™ (GibcoBRL). This is referred to as the “template mix.” The template mix was combined with the Lipofectamine in a polystyrene tube and was incubated for 30 minutes. During the incubation, the cells were washed with 100 μl Optimem™. After incubation, 200 μl of Optimem™ was added to the mix; 40 μl-50 μl of this mixture were added to each well. The cells were left to mix overnight. The media was replaced with fresh medium the following morning to DMEM/Phenol red free/1% FBS at a volume of about 130 μl/well. The cells were then assayed for luciferase activity using a Luclite™ Kit (Packard, Cat. # 6016911) and Trilux 1450 Microbeta™ liquid scintillation and luminescence counter (Wallac) as per the manufacturer's instructions. The data were analyzed using GraphPad Prism™ 2.0a (GraphPad Software Inc.).

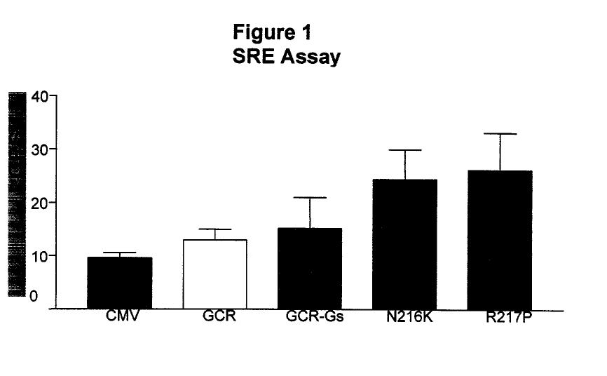

Reference is made to FIG. 1. In FIG. 1, when comparing the non-endogenous versions of plant GCR1 (“N216K” and “R217P”)) with the endogenous version (“wt”), the N216K and R217P versions both evidence about a 2 fold increase in relative light units compared with the endogenous version of plant GCR1 (“wt”). This data suggests that both non-endogenous versions of plant GCR1 (N216K and R217P) are constitutively activated.

### Example 5

### Fusion Protein Preparation: GPCR:Gs Fusion Construct

The design of the GPCR-Gs fusion protein construct was accomplished as follows: both the 5′ and 3′ ends of the rat G protein Gsα (long form; Itoh, H. et al., 83 PNAS 3776 (1986)) were engineered to include a HindIII (5′-AAGCTT-3′) sequence thereon. Following confirmation of the correct sequence (including the flanking HindIII sequences), the entire sequence was shuttled into pcDNA3.1(−) (Invitrogen, cat. no. V795-20) by subcloning using the HindIII restriction site of that vector. The correct orientation for the Gsα sequence was determined after subcloning into pcDNA3.1(−). The modified pcDNA3.1(−) containing the rat Gsα gene at HindIII sequence was then verified; this vector is now available as a “universal” Gsα protein vector. The pcDNA3.1(−) vector contains a variety of well-known restriction sites upstream of the HindIII site, thus beneficially providing the ability to insert, upstream of the Gs protein, the coding sequence of the plant GCR1. This same approach can be utilized to create other “universal” G protein vectors, and, of course, other commercially available or proprietary vectors known to the artisan can be utilized—the important criteria is that the sequence for the GPCR be upstream and in-frame with that of the G protein.

GCR1-Gsα Fusion Protein construct was then made as follows:

| | |
|---|---|
| 5′-TCTAGACCCGGGATGTCGGCGGTTCTCACA-3′ | (SEQ.ID.NO.:13; sense) |
| 5′-CAAGCTTGGTACCGATTGCTGGTCCTCGGTCTTGAGTGA-3′. | (SEQ.ID.NO.:14; antisense) |

The sense and anti-sense primers included the restriction sites for XbaI and KpnI, respectively.

PCR was then utilized to secure the respective receptor sequences for fusion within the Gsα universal vector disclosed above. The PCR reaction was performed in 100 μl containing 50 pmol of each primer, 0.4 mM DNA and 1.0 unit Pfu polymerase (Stratagene). Reaction temperatures and 30 cycle times for GCR1 were as follows: 94° C. for 40 seconds, 48° C. for 40 seconds; 72° C. for 3 minutes; 72° C. for 10 minutes.

The resulting 1,000 bp PCR product was run on a 1% agarose gel and then purified with QIAquick Gel Extraction kit (QIAGEN, used according to manufacturer's instructions). The purified product was digested with XbaI and KpnI (New England Biolabs) and the desired inserts were isolated, purified and ligated into the Gs universal vector at the respective restriction site. The positive clones were isolated following transformation and determined by restriction enzyme digest; expression using 293 cells was accomplished following the protocol set forth infra. Each positive clone for GCR1:Gs—Fusion Protein was sequenced and made available for the direct identification of candidate compounds. Nucleic acid (SEQ.ID.NO.:15) and amino acid (SEQ.ID.NO.:16) sequences for plant GCR1:Gs Fusion Protein were thereafter determined.

### Example 6

### GCR1:Gα Chimera Protein Preparation

The design of the GPCR-Gα chimera was accomplished by initially isolating and synthesizing the plant G protein-alpha (“Gα”) subunit. Total RNA was prepared from Arabidopsis floral organs at different developmental stages utilizing the Trizol Reagent (Life Technologies) according to the manufacturer's instructions. Poly (A+) RNA was obtained using the PolyATract® mRNA Isolation System (Promega) used according to manufacturer's instructions. Avian Myeloblastosis Virus Reverse Transcriptase (Promega) was used to generate cDNA from 0.5-3.0 μg of Poly (A+) or total RNA respectively, using 1 μg of oligo (dT) in the standard reaction conditions (Sambrook et al., Molecular Cloning: A Laboratory Manual, Cold Spring Harbor Press, 1989).

Based upon the genomic nucleotide sequence published in Genbank™ (Accession No. AC074309), two oligonucleotides primers were designed and had the following sequence:

| | |
|---|---|
| 5′-ATGGGCTTACTCTGCAGTAGAAG-3′ | (SEQ.ID.NO.:17) |
| 5′-GAGGTAATTTACTAGAGATATG-3′. | (SEQ.ID.NO.:18) |

The resulting 1,150 bp fragment was amplified by 30 cycles of PCR, each cycle comprising the following: 94° C. for 40 seconds, 50° C. for 40 seconds, 72° C. for 2 minutes, and an extension step at 72° C. for 10 minutes. The reaction was performed in 50 μl containing 30 pmol of each primer, 0.2 mM DNA and 1.0 unites of Pfu polymerase (Stratagene) in the corresponding buffer.

3′ A-overhangs were added and cloned into a pCR2.1 Topo® vector, an additional 10 minutes extension was performed after adding 0.5 units of Ampli-Taq® polymerase (Perkin Elmer). The 1,150 bp fragment corresponding to Gα open reading frame was cloned into a pCR2.1 Topo® (Invitrogen) and sequenced by Sequenase™ Dideoxy Chain Termination (United States Biochemical).

Recombinant PCR (Higuchi et al., Genomics. 1990, 6(1):65-71.) was then performed to fuse the plant GCR1 to the plant Gα subunit. Two PCR reactions were carried out separately on the full cDNAs using following two primers that produce two products with an overlapping region:

| | |
|---|---|
| 5′-AAGAACGAGGACCAGCAAATGGGCTTACTCT | (SEQ.ID.NO.:19) |
| GCAGT-3′ and | |
| 5′-ACTGCAGAGTAAGCCCATTTGGCTGGTCCTC | (SEQ.ID.NO.:20) |
| GTTCTT-3′. | |

The overlapping primary products were subsequently re-amplified using the flanking primers, having the following sequences:

| | |
|---|---|
| 5′-TCTAGACCCGGGATGTCGGCGGTTCTCACA | (SEQ.ID.NO.:21) |
| -3′ and | |
| 5′-AGCCATATGTAAAAGGCCAGCCTCCAG-3′ | (SEQ.ID.NO.:22) |

resulting in a recombinant fragment containing both sequences fused in frame. The flanking primers were designed to generate XbaI and SmaI restriction sites at the 5′ end and a NdeI restriction at the 3′ end. The primary PCR reactions were performed in 50 μl containing 30 pmol of each primer, 0.2 mM DNA and 1.0 units of Pfu polymerase (Stratagene) in the corresponding buffer. The amplification was obtained by 30 cycles of Polymerase chain reaction (PCR) each cycle comprising 94° C. for 40 seconds, 50° C. for 40 seconds, 72° C. for 2 minutes, and an extension step at 72° C. for 10 minutes.

The primary PCR products were then purified with the QIAquick® PCR Purification Kit (QIAGEN) used according to the manufacturer's instructions, and amplified together in a single second reaction. For the second PCR reaction, the following primers were utilized:

| | |
|---|---|
| 5′-TCTAGACCCGGGATGTCGGCGGTTCTCACA | (SEQ.ID.NO.:21) |
| -3′ and | |
| 5′-AGCCATATGTAAAAGGCCAGCCTCCAG-3′. | (SEQ.ID.NO.:22) |

The secondary PCR reaction was performed in 100 μl containing 50 pmol of each primer, 0.4 mM DNA and 1.0 Pfu polymerase (Stratagene) in the corresponding buffer. The recombinant fragment was amplified in 12 cycles using the same conditions reported above. The recombinant mutated product was digested with XbaI and NdeII (Gibco BRL) and cloned in the corresponding sites of the pET21a (Novagen) giving rise to the pET GCR-Gα and sequenced by Sequenase™ Dedeoxy chain termination (United States Biochemical). Nucleic acid (SEQ.ID.NO.:23) and amino acid (SEQ.ID.NO.:24) sequences for plant GCR1:Gα Chimera Protein were thereafter determined and verified.

### Example 7

### Transformation and Regeneration of Transgenic Plants

A. Plant Growth

Pots were prepared with soil (loosely packed) and covered with a window screen mesh. The mesh was placed in contact with the soil surface to allow the seedlings to germinate through. Five 2¼ inch. square pots were used to plant the seeds (ecotypes Columbia), having several seeds per pot. After one week of germination, one seedling at each spot was chosen and removed from the rest. The plants were grown to a stage at which bolts were emerging, for example, about 1 cm tall. The tip of the plants were cut off from the emerging plant bolt to induce the growth of the secondary inflorescences. The cut was made above the top most cauline leaf, leaving the axillary inflorescence meristems at the base of the cauline leaves. The plants were watered on the third day following decaptiation. Infiltration (see, Example 7(B) below) was performed about 4 days after decapitation of the plant. Developing siliques were then removed and the flowers were fertilized. The soil contained enough water at the time of infiltration so that it would not absorb much of the Agrobacterium tumefaciens suspension.

Agrobacterium tumefaciens were transformed with the pMON-GCR1 plasmid and used to produce transgenic BY-2 cell lines. BY-2 cells were transformed according to the method disclosed in An, G. Plan Physiol. 79, 568-570 (1985). The selected transgenic alli were grown in the dark on selective medium at the 24° C. and transferred to fresh medium every 4 week. Liquid cultures were obtained for the different transgenic lines and synchronization was achieved by a 24-h subculture in 5 mg/liter aphidicolin. See, Combettes, B. et al., Methods Cells Sci., 21, 109-121 (1991).

B. Infiltration

A large liquid culture of Agrobacterium tumefaciens C-58 AGL-O was grown to carry the plasmids containing the endogenous and non-endogenous versions of the GCR1 receptor. A 10 ml preculture was prepared followed by inoculation of a large culture of medium (½× Murashige & Skoog medium with Gamorg's vitamins; 5% sucrose, 0.5 g MES, and pH 5.7 with KOH, (autoclaved and allowed to cool), just before using the medium the following was added: 0.044 μM benzylaminopurine and 0.02% Silwet L-77) with antibiotics the day before infiltration. The cells were then harvested by centrifugation (5K, 15 minutes, room temperature) and resuspended in infiltration medium to an OD₆₀₀ of approximately 0.8. A 200 ml suspension was required for each infiltration. The same suspension was reused as many as three times. Two hundred ml of Agrobacterium tumefaciens suspension was added to the beaker. A pot containing plants was inverted into the suspension such that the entire plant was submerged. The plant was submerged for about 15 minutes. The pots were then placed on their side in a plastic flat and covered with a plastic dome to maintain humidity. The following day, the plastic flat was uncovered and the pots were set upright. The pots were not watered for several days. After this time, a small amount of water was added to the plastic flat. The plants were allowed to grow for 3 to 4 weeks, keeping the bolts from each pot together and separated from the neighboring pots. The seeds were then harvested.

C. Selection of Transformants

Plates were prepared for selection with the following: 1× Murashige & Skoog medium with Gamorg's vitamins; 1% sucrose, 0.5 g MES, and pH 5.7 with KOH, autoclaved, cool to 50° C., and just before using the medium 25 μmg/ml of kanamycin was added. The plates were dried well in the hood before planting the seeds. The seeds were then surface-sterilized and treated with 70% ethanol for 2 minutes, 5% bleach/94% water/1% SDS for 15 minutes, followed by three rinses with sterile water. The seeds were then resuspended in 1 ml sterile water in a 15 ml sterile tube. Six ml of cool top agar (0.1% tissue culture agar containing 100 mg/100 ml of H₂O, (autoclaved for 15 minutes and was then allowed to cool) was then added. The mixture was then poured onto the plate and gently rocked to let the liquid spread over the media. The plate was then allowed to sit in the hood with the fan on for 30 minutes, and was rotated 180° for an additional 30 minutes. The plate was then sealed with Parafilm. Up to 100 μl of dry seeds were planted per plate.

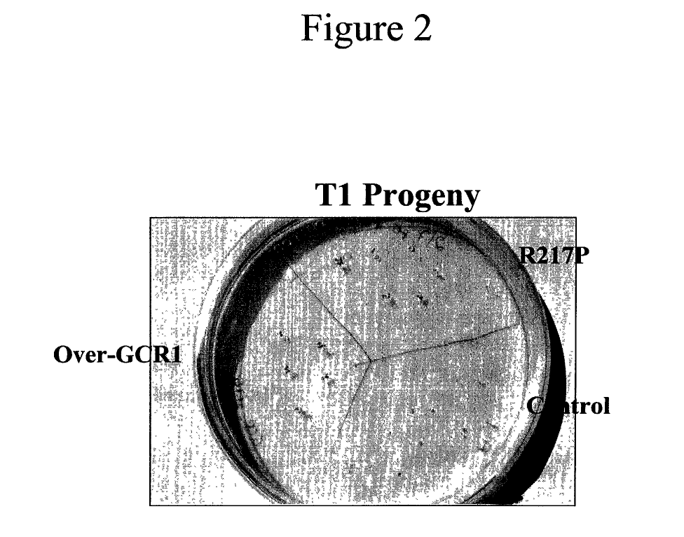

The plates containing the endogenous versions of the GCR1 was then left for 2-7 days in the cold room, followed by incubation in the growth room. For maturation of seeds, plates would normally be placed in a cold room for 2-7 days, followed by incubation in a growth room (for germination). However, in this experiment, plates were not placed in either a cold room or in a growth room, but rather left at room temperature. FIG. 2 depicts a plate containing seedlings without receptor (“Control”), seedlings with over-expressed endogenous GCR1 (“over-GCR1”), and seedlings with non-endogenous version of GCR1 (“R217P”). This data suggests that over-expressed GCR1 and non-endogenous version of GCR1 can mature without having to undergo a germination period. Therefore, an agonist against this non-endogenous, constitutively active version of GCR1 is expected to be useful in, for example, fruit and vegetable ripening and development, while an inverse agonist to the receptor is expected to be useful as, for example, a herbicide.

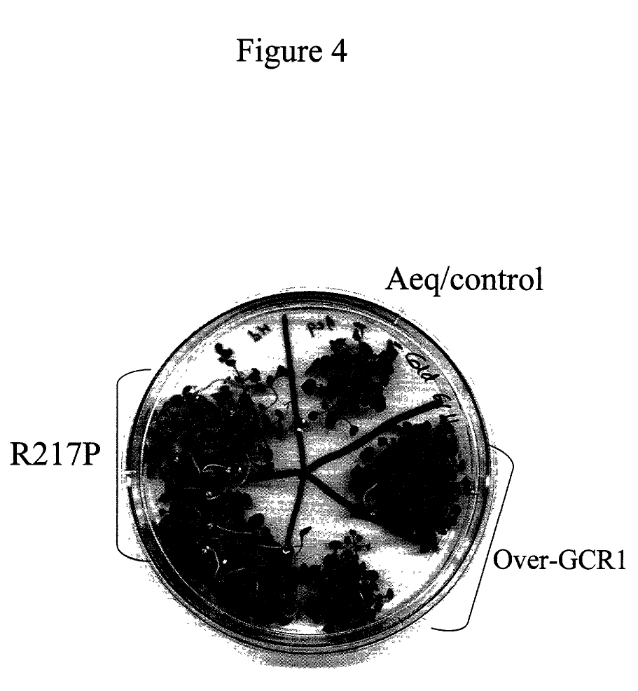

FIG. 4 a photograph of a plate containing buds of T3 Arabidopsis. FIG. 4 shows T3 progeny of overexpressed GCR1 (“over-GCR1”) and non-endogenous version GCR1 (“R217P”). FIG. 4 evidences that the non-endogenous version of GCR1 (R217P) and overexpressed GCR1 stimulate the growth of flowers in the plant. This data suggest that the “over-GCR1” and non-endogenous GCR1 (R217P) leads to early flowering by turning on the meristem identity gene LEAFY (“LFY”), which is known to be a transcription factor expressed throughout the flower.

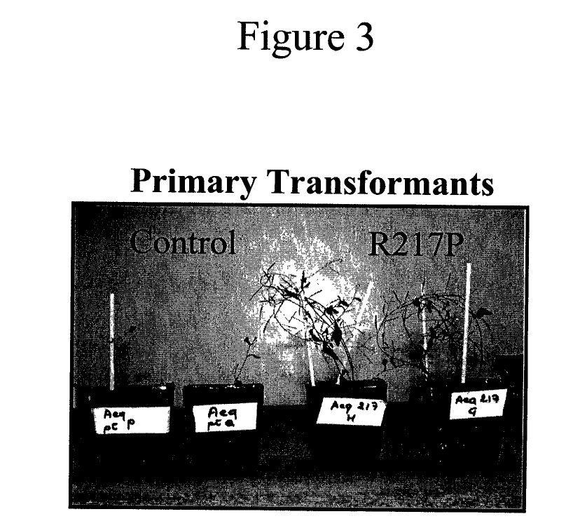

After 5 to 10 days, transformants were easily identified as dark green plants with long roots. These seedlings were then transferred to soil and kept covered for several days. FIG. 3 is a comparison of plants containing Aequorin (a control which measures calcium) with Aequorin plus non-endogenous version of GCR1 (“R217P”). FIG. 3 evidences that the non-endogenous version of GCR1 (R217P) significantly increased both in length and number of branches of the plant compared to the plants without the R217P version (i.e., the R217P version evidenced a phenotypic change). This phenotypic change supports the position that non-endogenous version of GCR1, R217P, is constitutively active. Therefore, an agonist against this receptor would be useful in, for example, stimulating the growth of fruit or vegetables, while an inverse agonist would be useful in, for example, inhibiting growth of plants, such as weeds, and thus utilized as a herbicide. Direct identification of a small molecule agonist can be amplified using the techniques disclosed herein. Such a molecule that acts as an agonist against the endogenous version of the receptor would positively address the public concerns regarding genetically altered plants.

### Example 8

### RT-PCR: Confirmation of Genes Expressed in GCR1:

RT-PCR was performed on total RNA and was prepared from Arabidopsis leaves and floral buds at different developmental stages utilizing the Trizol™ Reagent (Life Technologies) according to the manufacturer's instructions. Avian Myeloblastosis Virus Reverse Transcriptase (Promega) was used to generate cDNA from 0.5-3.0 μg of Poly (A⁺) or total RNA respectively, using 1 μg of oligo (dT) in the standard reaction conditions (Maniatis, 1989). Oligonucleotides used for PCR had the following specific sequences:

| Genes | 5′ Oligonucleotide (SEQ.ID.NO.) | 3′ Oligonucleotide (SEQ.ID.NO.) |
|---|---|---|
| LEAFY | GACGCCGTCATTTGCTACTCTC (25) | CGTCGTCATCCTCACCTTCGTT (26) |
| PP2A | GATGAGCAGATCTCGCAG (27) | TCCTTGACCAAATGTATATCC (28) |
| AtMYB65 | CACGGCGAGGGTAACTGGAA (29) | TGAAGGGAGCTCCAGCTTCA (30) |

The resulting fragment was amplified by 30 cycles of Polymerase chain reaction (PCR) each cycle comprising 30 sec at 94° C., 30 sec at 48° C., 45 sec at 72° C., and an extension step at 72° C. for 10 min. The reaction was performed in 50 μl containing 30 pmol of each primer, 0.2 mM deoxyribonucleotides and 1.0 unit of Pfu polymerase (Stratagene) in the corresponding buffer.

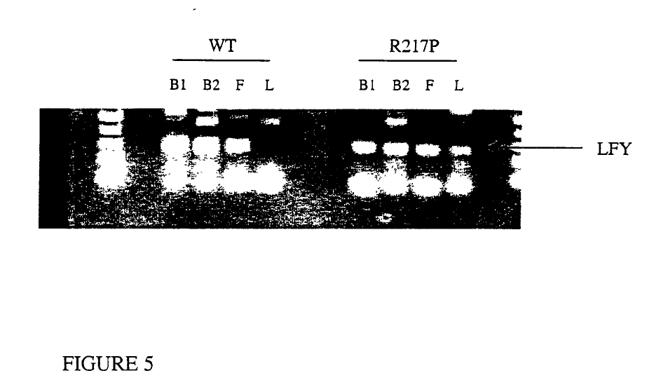

Flowering is another aspect of Arabidopsis development that is regulated by the plant hormone, GA. Arabidopsis is a long-day plant that bolts and flowers as a result of the synthesis of active Gαs under the proper day length conditions. Under short days, flowering is delayed. In FIG. 5, the fragment corresponding to the chosen leafy sequence was amplified in the buds (“B1” and “B2”) and flowers (“F”) of both wild-type plants and R217P version of GCR1. However, in amplification of the LEAFY protein was only expressed in the non-endogenous version of GCR1 (R217P) and not the control (wild-type GCR1). This data supports the position that the non-endogenous, constitutively activated version of GCR1 (R217P) results in early flowering of the Arabidopsis plant.

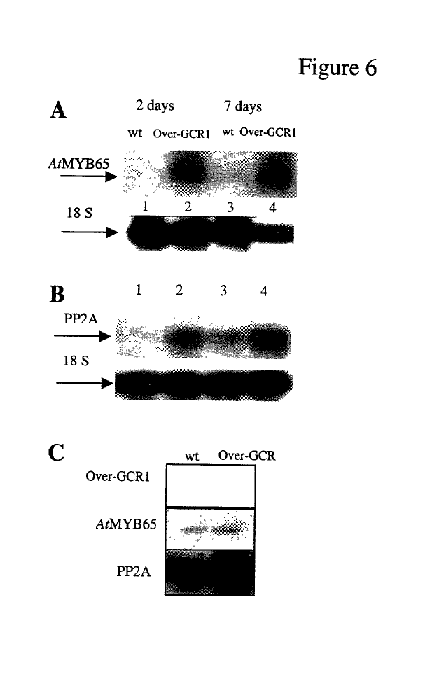

Seed dormancy and germination are regulated by gibberellin (GA) and abscisic acid (ABA) and release from dormancy is accompanied by changes in expression of many genes. In a number of species including Arabidopsis, seed dormancy can be overcome by GA. In barley (Hordeum vulgare), where the control of germination by GA and GA-induced gene expression have been studied for many years, the expression of such genes is dependent on the transcription factor GAMYB. The expression of GAMYB is itself GA-dependent. To find the closest Arabidopsis homologue of GAMYB, a Blast search was performed on the Arabidopsis database and identified MYB9, which is identical to AtMYB65. When the expression of AtMYB65 in wild-type was compared with that in GCR1 transformed plants after 2 and 7 days of germination, we found high expression of AtMYB65 in the transformants, but not in wild-type plants (FIG. 6a).

It was previously reported that release from dormancy in beechnut was accomplished by the activation of the gene encoding the catalytic subunit of the serine/threonine phosphatase PP2A (see, Gonzales-Pa, M. et al., Proceedings of The 17th International Conference on Plant Growth Substances, Bmo (Czech Republic) Abstract No. 3 (2001)). Utilizing RT-PCR, it was determined that no detectable expression of the gene for the catalytic subunit of PPA2 in the wild-type seeds (“wt”) seven (7) days after sowing, but expression was easily detected in the GCR1 transformants at day seven. See FIG. 6B. However, two weeks following sowing, when a substantial number of wild-type seeds had formed plantlets, expression of GCR1, AtMYB65 and the PPA2 catalytic subunit gene in the wild-type and GCR1 transformants were detected. See, FIG. 6C.

These results are consistent with the interpretation that seeds of the transformants have lost dormancy because they are more responsive to their endogenous Gαs and/or less sensitive to their endogenous ABA.

### Example 9

### Germination in Sucrose

Seeds were sterilized in 70% ethanol for 2 minutes, and 5% bleach for 30 minutes. The seeds were then placed on ½ MS media with varying amounts of sucrose (e.g., 33 mM and 330 mM).

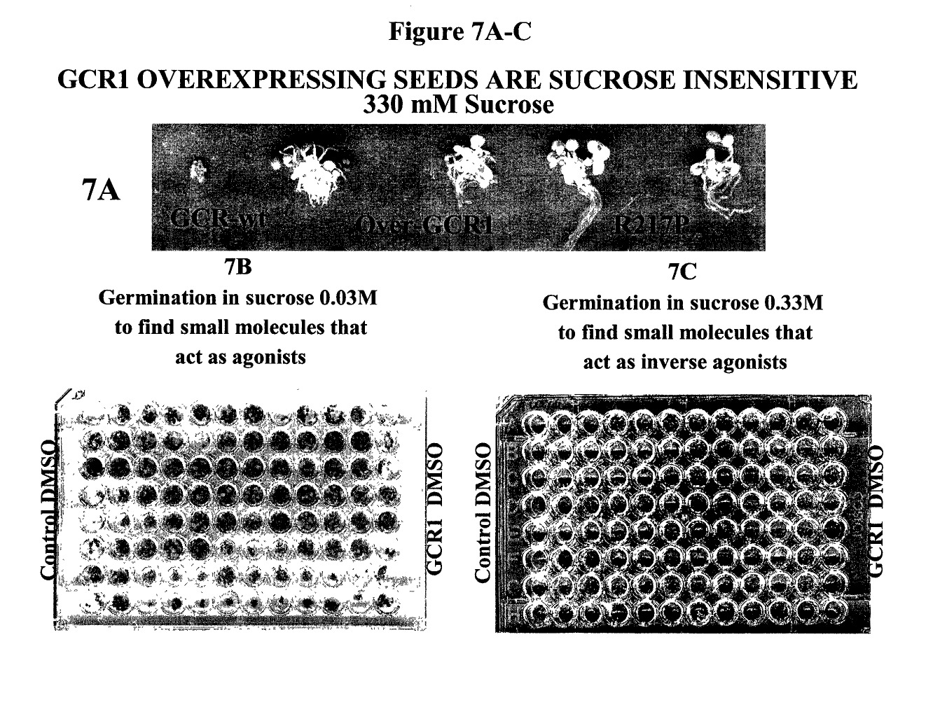

Reference is made to FIGS. 7A-C. FIG. 7A depicts the expression of GCR1 seedlings in the presence of 330 mM sucrose. This data evidences that the seedlings with wild-type GCR1 (“GCR1-wt”) were not able to germinate in the presence of 330 mM of sucrose. However, seedlings with overexpressed GCR1 (“Over-GCR1”) and the constitutively activated version of GCR1 (“R217P”) both seedlings germinated, with the R217P version evidencing longer roots. Typically, seedlings will not germinate in the presence of 330 mM sucrose. This data suggests that overexpressed GCR1 and the non-endogenous, constitutively active version of GCR1 (R217P) can mature in the presence of sucrose. With this data, the present invention relates, inter alia, to a method useful in identifying an agonist (see FIG. 7B) useful in, for example, fruit and vegetable ripening and development, while an inverse agonist (see FIG. 7C) is useful as, for example, a herbicide against the GCR1 receptor.

### Example 10

### Thymidine Incorporation and Mitotic Index

DNA synthesis is measured by incubating 1 ml samples of the synchronized cell suspension with 1 μCi of [³H] thymidine for 20 minutes at 27° C. in Eppendorf microtubes with gentle shaking. Cells were collected by centrifugation for 5 minutes at 1,800× g, and the pellet was frozen in dry ice. DNA was extracted with Plant DNAzol Reagent (GIBCO/BRL), resuspended in water, and the [³H] radioactivity was determined with a scintillation counter.

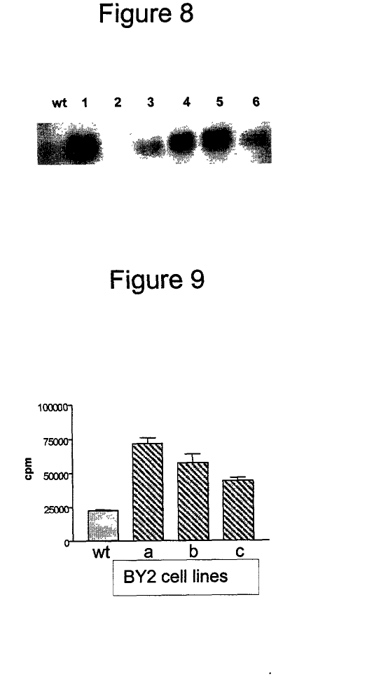

The modulation of GCR1 during the cell cycle indicated to that it might be instructive to determine the effect of overexpression of GCR1 on the cell cycle and on the expression of cell cycle associated genes. For this line of experiments, a tobacco (Nicotiana tabacum) cell line (BY-2) was utilized, a cell line that is easily synchronizable and used extensively for cell cycle studies. Transformation of BY-2 cells with the Arabidopsis GCR1 gene driven by the CaMV25S promoter yielded cell lines that had different levels of GCR1 mRNA as determined by Northern blot. (See, FIG. 8).

A GTPγS binding assay was then performed to determine if the transformed cell lines contained higher levels of active receptor. This assay used microsomes and three cell lines that had the highest level of GCR1 expression. After GPCR activation and G-protein coupling, the level of activation was measured using GTPγ[³⁵S] binding because GTPγ[³⁵S] cannot be hydrolyzed to GDP. The bound radioactivity is a measure of the abundance of Gα bound to active GPCR. The amount of GTPγS bound to microsomes from the transformed BY-2 lines was 2 to 3 times higher than that bound to microsomes from wild-type BY-2 cells (FIG. 9). These data evidence that the transformed cells contained more active GCR1 than wild-type cells and that this GCR1 couples to Gα.

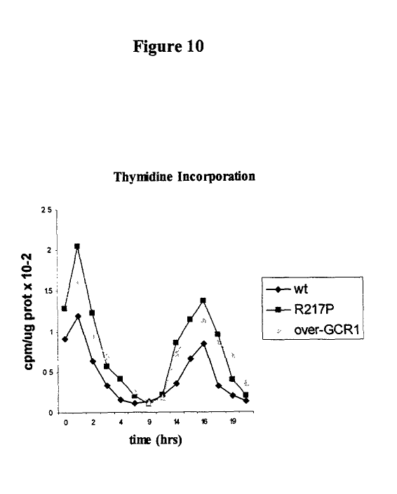

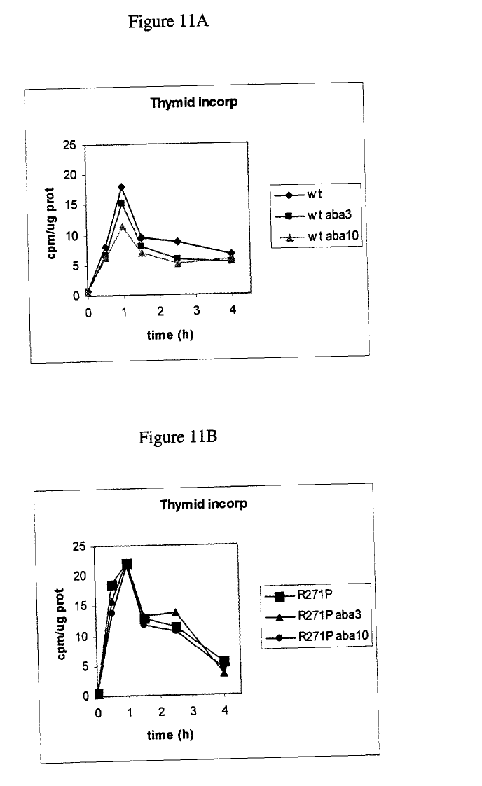

To determine the effect of the non-endogenous, constitutively activated version of GCR1, R217P, on the cell cycle, control cell (empty vector), wild-type GCR1 (“wt”) and R271P overexpressing BY-2 cells were synchronized with aphidicolin for 24 h, and, after removal of the cell cycle blocker, thymidine incorporation was measured. (See, FIG. 10).

Reference is made to FIG. 10. In FIG. 10, wt GCR1 BY-2 cells, thymidine incorporation showed two clear peaks: one at 1 hr after removal of the aphidicolin and a second one at 16 hr. This result is consistent with a block by aphidicolin in early S-phase so that DNA synthesis starts immediately after the removal of the block. The cell cycle under these conditions is about 14-15h. In the GCR1 overexpressing cells and more in the R271P activated GCR1 there was a considerable increase in thymidine incorporation, suggesting that the rate of DNA synthesis may be enhanced in these cells.

Reference is made to FIGS. 11A-B. In FIGS. 11A-B, thymidine incorporation into BY-2 cells were measured over time comparing the wild-type GCR1 (“wt”) (FIG. 11A) and the non-endogenous, constitutively activated version of GCR1, R271P, (FIG. 11B) in the presence of 3 μM and 10 μM Abscissic Acid (“ABA”). ABA is a plant hormone related to plant stress response. Based upon data presented in FIG. 11A, ABA decreases thymidine incorporation of wild-type GCR1. In other words, DNA synthesis of the wild-type GCR1 in BY-2 cells was reduced. FIG. 11B evidences that DNA synthesis of R271P is not affected by the presence of ABA. In other words, constitutively activated version of GCR1, R271P, is insensitive to the plant hormone ABA.

While not wishing to be bound to any theory, it is believed that based upon the data presented throughout this patent document, GCR1 may play a role in regulating the cell cycle.

References cited throughout this patent document, including co-pending and related patent applications, unless otherwise indicated, are fully incorporated herein by reference in its entirety. Modifications and extension of the disclosed inventions that are within the purview of the skilled artisan are encompassed within the above disclosure and the claims that follow.

Although a variety of expression vectors are available to those in the art, for purposes of utilization for both the endogenous and non-endogenous GPCRs, it is most preferred that the vector utilized be pCMV. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be viable. The ATCC has assigned the following deposit number to pCMV: ATCC #203351.

What is claimed is:

1. A non-endogenous, constitutively activated plant G protein-coupled receptor (GPCR).

2. The plant GPCR of claim 1 having an amino acid sequence selected from the group consisting of SEQ ID NOS:10 and 12.

3. The plant GPCR of claim 1 encoded by a nucleotide sequence selected from the group consisting of SEQ ID NOS: 9 and 11.

4. A plasmid comprising a vector and a cDNA selected from the group consisting of SEQ.ID.NOS:9 and 11.

5. A host cell comprising the plasmid of claim 4.

6. A method for directly identifying a non-endogenous candidate compound as an agonist or an inverse agonist to an endogenous plant GPCR, said method comprising the steps of:
(a) subjecting said endogenous plant GPCR to constitutive receptor activation to create a non-endogenous, constitutively activated plant GPCR;
(b) contacting the non-endogenous candidate compound with said non-endogenous, constitutively activated plant GPCR; and
(c) identifying said non-endogenous candidate compound as an inverse agonist or an agonist to said constitutively activated plant GPCR by measuring at least a 20% difference in an intracellular signal induced by said contacted compound as compared with an intracellular signal in the absence of said contacted compound.

7. A method for directly identifying a non-endogenous candidate compound as an agonist or an inverse agonist to an endogenous constitutively activated plant GPCR, said method comprising the steps of:
(a) contacting the non-endogenous candidate compound with said endogenous constitutively activated plant GPCR; and
(b) identifying said non-endogenous candidate compound as an inverse agonist or an agonist to said endogenous constitutively activated plant GPCR by measuring at least a 20% difference in an intracellular signal induced by said contacted compound as compared with an intracellular signal in the absence of said contacted compound.

8. The method of either claim 6 or 7 wherein an endogenous ligand for said endogenous plant GPCR has not been identified.

9. A compound identified by the method of claim 6 or 7.

10. A composition comprising the compound of claim 9.

11. A method of modulating a physiological process in a plant comprising subjecting an endogenous plant GPCR to constitutive receptor activation to create a non-endogenous, constitutively activated plant GPCR, said physiological process thereby being modulated.

12. A method of modulating a physiological process in a plant comprising:
(a) subjecting an endogenous plant GPCR to constitutive receptor activation to create a non-endogenous, constitutively activated plant GPCR; and
(b) contacting said non-endogenous, constitutively activated plant GPCR with a non-endogenous agonist or inverse agonist of said GPCR,
said physiological process thereby being modulated.

13. The method of either of claim 11 or 12 wherein said endogenous plant GPCR has an amino acid sequence selected from the group consisting of SEQ ID NOS: 10 and 12.

14. The method of either of claim 11 or 12 wherein said plant is selected from the group consisting of roses, daffodils, tobacco, carnation, freesia, cotton, geranium, iris, chrysanthemum, lily, orchid, sunflower, and tulips.

15. The method of claim 14 wherein said plant is rose.

16. The method of either of claim 11 or 12 wherein said physiological process is selected from the group consisting of herbicide sensitivity, germination, growth elongation, seed dormancy, drought tolerance, pesticide sensitivity, quiescence, cell-cycle regulation, flower development, and fruit and vegetable ripening and development.

17. A method for directly identifying a non-endogenous candidate compound as a compound having activity selected from the group consisting of inverse agonist activity and agonist activity, to an endogenous, constitutively active plant G protein coupled cell surface receptor (GPCR) comprising the steps of:
(a) contacting a non-endogenous candidate compound with a GPCR Fusion Protein, said GPCR Fusion Protein comprising said endogenous, constitutively active plant GPCR and a G protein; and
(b) identifying said non-endogenous candidate compound as an inverse agonist or an agonist to said endogenous constitutively activated plant GPCR by measuring at least a 20% difference in an intracellular signal induced by said contacted compound as compared with an intracellular signal in the absence of said contacted compound.

18. A method for directly identifying a non-endogenous candidate compound as a compound having activity selected from the group consisting of inverse agonist activity and agonist activity, to an endogenous, constitutively active plant G protein coupled cell surface receptor (GPCR) comprising the steps of:
(a) contacting a non-endogenous candidate compound with a GPCR Fusion Protein, said GPCR Fusion Protein comprising said endogenous, constitutively active plant GPCR and a G protein; and
(b) determining whether a receptor functionality is modulated, wherein a change in receptor functionality is indicative of the candidate compound being an agonist or inverse agonist of said endogenous, constitutively active plant GPCR.

19. The method of either of claim 17 or 18 wherein the endogenous ligand for said endogenous, constitutively active plant GPCR has not been identified.

20. A GPCR Fusion Protein construct comprising a constitutively active plant G protein coupled receptor and a G protein.

21. The GPCR Fusion Protein construct of claim 20 wherein said constitutively active plant GPCR is non-endogenous.

22. The GPCR Fusion Protein construct of claim 20 wherein said constitutively active plant G protein coupled receptor comprises an amino acid sequence selected from the group consisting of SEQ.ID.NOS.:10 and 12.

23. The GPCR Fusion Protein construct of claim 20 wherein said G protein is Gα.

24. The method of any one of claims 6, 7, 17, or 18 wherein said non-endogenous candidate compound is an agonist.

25. The method of any one of claims 6, 7, 17, or 18 wherein said non-endogenous candidate compound is an inverse agonist.

26. The method of claim 12 wherein said non-endogenous, constitutively activated plant GPCR is contacted with a non-endogenous agonist of said GPCR.

27. The method of claim 12 wherein said non-endogenous, constitutively activated plant GPCR is contacted with a non-endogenous inverse agonist of said GPCR.

28. A method for modulating a physiological process in a plant, said method comprising the steps of:
(a) subjecting an endogenous plant GPCR to constitutive receptor activation to create a non-endogenous, constitutively activated plant GPCR;
(b) contacting the non-endogenous candidate compound with said non-endogenous, constitutively activated plant GPCR;
(c) identifying said non-endogenous candidate compound as an inverse agonist or an agonist to said non-endogenous constitutively activated plant GPCR by measuring at least a 20% difference in an intracellular signal induced by said contacted compound as compared with an intracellular signal in the absence of said contacted compound; and
(d) contacting said plant with said inverse agonist or agonist;
the physiological process in said plant thereby being modulated.

29. A method for modulating a physiological process in a plant, said method comprising the steps of:
(a) subjecting an endogenous plant GPCR to constitutive receptor activation to create a non-endogenous, constitutively activated plant GPCR;
(b) contacting the non-endogenous candidate compound with said non-endogenous, constitutively activated plant GPCR to yield a plant comprising a non-endogenous constitutively activated GPCR;
(c) identifying said non-endogenous candidate compound as an inverse agonist or an agonist to said non-endogenous constitutively activated plant GPCR by measuring at least a 20% difference in an intracellular signal induced by said contacted compound as compared with an intracellular signal in the absence of said contacted compound; and
(d) contacting said plant comprising a non-endogenous constitutively activated GPCR with said inverse agonist or agonist;
the physiological process in said plant thereby being modulated.

30. The method of either of claim 28 or 29 wherein said physiological process is selected from the group consisting of herbicide sensitivity, germination, growth elongation, seed dormancy, drought tolerance, pesticide sensitivity, quiescence, cell-cycle regulation, flower development, and fruit and vegetable ripening and development.

31. A plant comprising a non-endogenous, constitutively activated plant G protein-coupled receptor (GPCR).

32. The plant of claim 31 wherein said non-endogenous, constitutively activated plant G protein-coupled receptor has an amino acid sequence selected from the group consisting of SEQ ID NOS:10 and 12.

33. The plant of claim 31 wherein said non-endogenous, constitutively activated plant G protein-coupled receptor is encoded by a nucleotide sequence selected from the group consisting of SEQ ID NOS: 9 and 11.

34. A plant contacted with an inverse agonist or agonist identified by either of claims 6 or 7.

35. The plant of claim 34 wherein said plant comprises a non-endogenous, constitutively activated plant G protein-coupled receptor (GPCR).

36. A plant contacted with an inverse agonist of a constitutively activated GPCR.


SEQUENCE LISTING (30 sequences)

SEQ ID NO: 1 - DNA, 981 bp, Arabidopsis thaliana
atgtcggcggttctcacagccggcggaggcttaactgccggggatcgcagcattattacggcgattaacaccggcgcttcgagtctctcattcgtcggctcagccttcatcgttctctgctactgcctcttcaaagaacttcgaaaattctctttcaagctcgtcttctaccttgctctctctgatatgctttgcagtttcttcctaatcgttggggatccttcaaaggggttcatttgttacgcacaaggctacactactcatttcttctgcgttgcttcgttcttgtggacaactacaattgctttcactcttcatcgtactgttgtcaagcataaaactgatgtggaggatttggaagcaatgtttcatttgtatgtatgggggacttccttggttgtgactgtcatacgttcttttggtaacaaccactcacatttggggccatggtgctggacgcaaactggtcttaagggaaaggctgttcattttttaactttctacgctcctctttggggagccattctttacaatgggtttacttacttccaagtgatacggatgctaagaaatgctagacgtatggcagttggaatgtcagaccgagtcgatcaatttgataatagagcagagttaaaggtgttgaacagatggggatactatccactcattctaataggatcatgggcattcggcactattaaccgtatccatgatttcatcgagccagggcataagatcttctggctctcagttcttgacgttgggacagctgcactaatgggcttgttcaattcaatagcctatggtttcaacagctcagtgcgtcgagcaatccatgagagactggagctattcttgccagaacggctatatcgatggcttccaagcaatttcagaccaaaaaaccatctgattctacatcagcaacaacaacagcgaagcgaaatggtatcactcaagaccgaggaccagcaatga

SEQ ID NO: 2 - protein, 326 aa, Arabidopsis thaliana
MSAVLTAGGGLTAGDRSIITAINTGASSLSFVGSAFIVLCYCLFKELRKFSFKLVFYLALSDMLCSFFLIVGDPSKGFICYAQGYTTHFFCVASFLWTTTIAFTLHRTVVKHKTDVEDLEAMFHLYVWGTSLVVTVIRSFGNNHSHLGPWCWTQTGLKGKAVHFLTFYAPLWGAILYNGFTYFQVIRMLRNARRMAVGMSDRVDQFDNRAELKVLNRWGYYPLILIGSWAFGTINRIHDFIEPGHKIFWLSVLDVGTAALMGLFNSIAYGFNSSVRRAIHERLELFLPERLYRWLPSNFRPKNHLILHQQQQQRSEMVSLKTEDQQ

SEQ ID NO: 3 - DNA, 30 bp, Artificial Sequence
  Synthetic Construct
tctagacccgggatgtcggcggttctcaca

SEQ ID NO: 4 - DNA, 39 bp, Artificial Sequence
  Synthetic Construct
gagttaaaggtgttgaagagatggggatactatccactc

SEQ ID NO: 5 - DNA, 36 bp, Artificial Sequence
  Synthetic Construct
tggatagtatccccatctcttcaacacctttaactc

SEQ ID NO: 6 - DNA, 31 bp, Artificial Sequence
  Synthetic Construct
atgatctcgagtcaaccctttgctgtccacc

SEQ ID NO: 7 - DNA, 36 bp, Artificial Sequence
  Synthetic Construct
ttaaaggtgttgaacccatggggatactatccactc

SEQ ID NO: 8 - DNA, 35 bp, Artificial Sequence
  Synthetic Construct
atagtatccccatggcttcaacacctttaactctc

SEQ ID NO: 9 - DNA, 981 bp, Artificial Sequence
  Synthetic Construct
atgtcggcggttctcacagccggcggaggcttaactgccggggatcgcagcattattacggcgattaacaccggcgcttcgagtctctcattcgtcggctcagccttcatcgttctctgctactgcctcttcaaagaacttcgaaaattctctttcaagctcgtcttctaccttgctctctctgatatgctttgcagtttcttcctaatcgttggggatccttcaaaggggttcatttgttacgcacaaggctacactactcatttcttctgcgttgcttcgttcttgtggacaactacaattgctttcactcttcatcgtactgttgtcaagcataaaactgatgtggaggatttggaagcaatgtttcatttgtatgtatgggggacttccttggttgtgactgtcatacgttcttttggtaacaaccactcacatttggggccatggtgctggacgcaaactggtcttaagggaaaggctgttcattttttaactttctacgctcctctttggggagccattctttacaatgggtttacttacttccaagtgatacggatgctaagaaatgctagacgtatggcagttggaatgtcagaccgagtcgatcaatttgataatagagcagagttaaaggtgttgaagagatggggatactatccactcattctaataggatcatgggcattcggcactattaaccgtatccatgatttcatcgagccagggcataagatcttctggctctcagttcttgacgttgggacagctgcactaatgggcttgttcaattcaatagcctatggtttcaacagctcagtgcgtcgagcaatccatgagagactggagctattcttgccagaacggctatatcgatggcttccaagcaatttcagaccaaaaaaccatctgattctacatcagcaacaacaacagcgaagcgaaatggtatcactcaagaccgaggaccagcaatga

SEQ ID NO: 10 - protein, 326 aa, Artificial Sequence
  Synthetic Construct
MSAVLTAGGGLTAGDRSIITAINTGASSLSFVGSAFIVLCYCLFKELRKFSFKLVFYLALSDMLCSFFLIVGDPSKGFICYAQGYTTHFFCVASFLWTTTIAFTLHRTVVKHKTDVEDLEAMFHLYVWGTSLVVTVIRSFGNNHSHLGPWCWTQTGLKGKAVHFLTFYAPLWGAILYNGFTYFQVIRMLRNARRMAVGMSDRVDQFDNRAELKVLKRWGYYPLILIGSWAFGTINRIHDFIEPGHKIFWLSVLDVGTAALMGLFNSIAYGFNSSVRRAIHERLELFLPERLYRWLPSNFRPKNHLILHQQQQQRSEMVSLKTEDQQ

SEQ ID NO: 11 - DNA, 981 bp, Artificial Sequence
  Synthetic Construct
atgtcggcggttctcacagccggcggaggcttaactgccggggatcgcagcattattacggcgattaacaccggcgcttcgagtctctcattcgtcggctcagccttcatcgttctctgctactgcctcttcaaagaacttcgaaaattctctttcaagctcgtcttctaccttgctctctctgatatgctttgcagtttcttcctaatcgttggggatccttcaaaggggttcatttgttacgcacaaggctacactactcatttcttctgcgttgcttcgttcttgtggacaactacaattgctttcactcttcatcgtactgttgtcaagcataaaactgatgtggaggatttggaagcaatgtttcatttgtatgtatgggggacttccttggttgtgactgtcatacgttcttttggtaacaaccactcacatttggggccatggtgctggacgcaaactggtcttaagggaaaggctgttcattttttaactttctacgctcctctttggggagccattctttacaatgggtttacttacttccaagtgatacggatgctaagaaatgctagacgtatggcagttggaatgtcagaccgagtcgatcaatttgataatagagcagagttaaaggtgttgaacccatggggatactatccactcattctaataggatcatgggcattcggcactattaaccgtatccatgatttcatcgagccagggcataagatcttctggctctcagttcttgacgttgggacagctgcactaatgggcttgttcaattcaatagcctatggtttcaacagctcagtgcgtcgagcaatccatgagagactggagctattcttgccagaacggctatatcgatggcttccaagcaatttcagaccaaaaaaccatctgattctacatcagcaacaacaacagcgaagcgaaatggtatcactcaagaccgaggaccagcaatga

SEQ ID NO: 12 - protein, 326 aa, Artificial Sequence
  Synthetic Construct
MSAVLTAGGGLTAGDRSIITAINTGASSLSFVGSAFIVLCYCLFKELRKFSFKLVFYLALSDMLCSFFLIVGDPSKGFICYAQGYTTHFFCVASFLWTTTIAFTLHRTVVKHKTDVEDLEAMFHLYVWGTSLVVTVIRSFGNNHSHLGPWCWTQTGLKGKAVHFLTFYAPLWGAILYNGFTYFQVIRMLRNARRMAVGMSDRVDQFDNRAELKVLNPWGYYPLILIGSWAFGTINRIHDFIEPGHKIFWLSVLDVGTAALMGLFNSIAYGFNSSVRRAIHERLELFLPERLYRWLPSNFRPKNHLILHQQQQQRSEMVSLKTEDQQ

SEQ ID NO: 13 - DNA, 30 bp, Artificial Sequence; identical to SEQ ID NO: 3
  Synthetic Construct

SEQ ID NO: 14 - DNA, 39 bp, Artificial Sequence
  Synthetic Construct
caagcttggtaccgattgctggtcctcggtcttgagtga

SEQ ID NO: 15 - DNA, 2163 bp, Artificial Sequence
  Synthetic Construct
atgtcggcggttctcacagccggcggaggcttaactgccggggatcgcagcattattacggcgattaacaccggcgcttcgagtctctcattcgtcggctcagccttcatcgttctctgctactgcctcttcaaagaacttcgaaaattctctttcaagctcgtcttctaccttgctctctctgatatgctttgcagtttcttcctaatcgttggggatccttcaaaggggttcatttgttacgcacaaggctacactactcatttcttctgcgttgcttcgttcttgtggacaactacaattgctttcactcttcatcgtactgttgtcaagcataaaactgatgtggaggatttggaagcaatgtttcatttgtatgtatgggggacttccttggttgtgactgtcatacgttcttttggtaacaaccactcacatttggggccatggtgctggacgcaaactggtcttaagggaaaggctgttcattttttaactttctacgctcctctttggggagccattctttacaatgggtttacttacttccaagtgatacggatgctaagaaatgctagacgtatggcagttggaatgtcagaccgagtcgatcaatttgataatagagcagagttaaaggtgttgaacagatggggatactatccactcattctaataggatcatgggcattcggcactattaaccgtatccatgatttcatcgagccagggcataagatcttctggctctcagttcttgacgttgggacagctgcactaatgggcttgttcaattcaatagcctatggtttcaacagctcagtgcgtcgagcaatccatgagagactggagctattcttgccagaacggctatatcgatggcttccaagcaatttcagaccaaaaaaccatctgattctacatcagcaacaacaacagcgaagcgaaatggtatcactcaagaccgaggaccagcaaatgggctgcctcggcaacagtaagaccgaggaccagcgcaacgaggagaaggcgcagcgcgaggccaacaaaaagatcgagaagcagctgcagaaggacaagcaggtctaccgggccacgcaccgcctgctgctgctgggtgctggagagtctggcaaaagcaccattgtgaagcagatgaggatcctacatgttaatgggtttaacggagagggcggcgaagaggacccgcaggctgcaaggagcaacagcgatggtgagaaggccaccaaagtgcaggacatcaaaaacaacctgaaggaggccattgaaaccattgtggccgccatgagcaacctggtgccccccgtggagctggccaaccctgagaaccagttcagagtggactacattctgagcgtgatgaacgtgccaaactttgacttcccacctgaattctatgagcatgccaaggctctgtgggaggatgagggagttcgtgcctgctacgagcgctccaacgagtaccagctgatcgactgtgcccagtacttcctggacaagattgatgtgatcaagcaggccgactacgtgccaagtgaccaggacctgcttcgctgccgcgtcctgacctctggaatctttgagaccaagttccaggtggacaaagtcaacttccacatgttcgatgtgggcggccagcgcgatgaacgccgcaagtggatccagtgcttcaatgatgtgactgccatcatcttcgtggtggccagcagcagctacaacatggtcatccgggaggacaaccagaccaaccgtctgcaggaggctctgaacctcttcaagagcatctggaacaacagatggctgcgtaccatctctgtgatcctcttcctcaacaagcaagatctgcttgctgagaaggtcctcgctgggaaatcgaagattgaggactactttccagagttcgctcgctacaccactcctgaggatgcgactcccgagcccggagaggacccacgcgtgacccgggccaagtacttcatccgggatgagtttctgagaatcagcactgctagtggagatggacgtcactactgctaccctcactttacctgcgccgtggacactgagaacatccgccgtgtcttcaacgactgccgtgacatcatccagcgcatgcatcttcgcgactgcgggctgttttaa

SEQ ID NO: 16 - protein, 720 aa, Artificial Sequence
  Synthetic Construct
MSAVLTAGGGLTAGDRSIITAINTGASSLSFVGSAFIVLCYCLFKELRKFSFKLVFYLALSDMLCSFFLIVGDPSKGFICYAQGYTTHFFCVASFLWTTTIAFTLHRTVVKHKTDVEDLEAMFHLYVWGTSLVVTVIRSFGNNHSHLGPWCWTQTGLKGKAVHFLTFYAPLWGAILYNGFTYFQVIRMLRNARRMAVGMSDRVDQFDNRAELKVLNRWGYYPLILIGSWAFGTINRIHDFIEPGHKIFWLSVLDVGTAALMGLFNSIAYGFNSSVRRAIHERLELFLPERLYRWLPSNFRPKNHLILHQQQQQRSEMVSLKTEDQQMGCLGNSKTEDQRNEEKAQREANKKIEKQLQKDKQVYRATHRLLLLGAGESGKSTIVKQMRILHVNGFNGEGGEEDPQAARSNSDGEKATKVQDIKNNLKEAIETIVAAMSNLVPPVELANPENQFRVDYILSVMNVPNFDFPPEFYEHAKALWEDEGVRACYERSNEYQLIDCAQYFLDKIDVIKQADYVPSDQDLLRCRVLTSGIFETKFQVDKVNFHMFDVGGQRDERRKWIQCFNDVTAIIFVVASSSYNMVIREDNQTNRLQEALNLFKSIWNNRWLRTISVILFLNKQDLLAEKVLAGKSKIEDYFPEFARYTTPEDATPEPGEDPRVTRAKYFIRDEFLRISTASGDGRHYCYPHFTCAVDTENIRRVFNDCRDIIQRMHLRQYELL

SEQ ID NO: 17 - DNA, 23 bp, Arabidopsis thaliana
atgggcttactctgcagtagaag

SEQ ID NO: 18 - DNA, 22 bp, Arabidopsis thaliana
gaggtaatttactagagatatg

SEQ ID NO: 19 - DNA, 36 bp, Artificial Sequence
  Synthetic Construct
aagaacgaggaccagcaaatgggcttactctgcagt

SEQ ID NO: 20 - DNA, 37 bp, Artificial Sequence
  Synthetic Construct
actgcagagtaagcccatttggctggtcctcgttctt

SEQ ID NO: 21 - DNA, 30 bp, Artificial Sequence; identical to SEQ ID NO: 3
  Synthetic Construct

SEQ ID NO: 22 - DNA, 27 bp, Artificial Sequence
  Synthetic Construct
agccatatgtaaaaggccagcctccag

SEQ ID NO: 23 - DNA, 2130 bp, Artificial Sequence
  Synthetic Construct
atgtcggcggttctcacagccggcggaggcttaactgccggggatcgcagcattattacggcgattaacaccggcgcttcgagtctctcattcgtcggctcagccttcatcgttctctgctactgcctcttcaaagaacttcgaaaattctctttcaagctcgtcttctaccttgctctctctgatatgctttgcagtttcttcctaatcgttggggatccttcaaaggggttcatttgttacgcacaaggctacactactcatttcttctgcgttgcttcgttcttgtggacaactacaattgctttcactcttcatcgtactgttgtcaagcataaaactgatgtggaggatttggaagcaatgtttcatttgtatgtatgggggacttccttggttgtgactgtcatacgttcttttggtaacaaccactcacatttggggccatggtgctggacgcaaactggtcttaagggaaaggctgttcattttttaactttctacgctcctctttggggagccattctttacaatgggtttacttacttccaagtgatacggatgctaagaaatgctagacgtatggcagttggaatgtcagaccgagtcgatcaatttgataatagagcagagttaaaggtgttgaacagatggggatactatccactcattctaataggatcatgggcattcggcactattaaccgtatccatgatttcatcgagccagggcataagatcttctggctctcagttcttgacgttgggacagctgcactaatgggcttgttcaattcaatagcctatggtttcaacagctcagtgcgtcgagcaatccatgagagactggagctattcttgccagaacggctatatcgatggcttccaagcaatttcagaccaaaaaaccatctgattctacatcagcaacaacaacagcgaagcgaaatggtatcactcaagaccgaggaccagcaaatgggcttactctgcagtagaagtcgacatcatactgaagatactgatgagaatacacaggctgctgaaatcgaaagacggatagagcaagaagcaaaggctgaaaagcatattcggaagcttttgctacttggtgctggggaatctggaaaatctacaatttttaagcagataaaacttctattccaaacgggatttgatgaaggagaactaaagagctatgttccagtcattcatgccaatgtctatcagactataaaattattgcatgatggaacaaaggagtttgctcaaaatgaaacagattctgctaaatatatgttatcttctgaaagtattgcaattggggagaaactatctgagattggtggtaggttagactatccacgtcttaccaaggacatcgctgagggaatagaaacactatggaaggatcctgcaattcaggaaacttgtgctcgtggtaatgagcttcaggttcctgattgtacgaaatatctgatggagaacttgaagagactatcagatataaattatattccaactaaggaggatgtactttatgcaagagttcgcacaactggtgtcgtggaaatacagttcagccctgtgggagagaataaaaaaagtggtgaagtgtaccgattgtttgacgtgggtggacagagaaatgagaggaggaaatggattcatctgtttgaaggtgtaacagctgtgatattttgtgctgccatcagcgagtacgaccaaacgctctttgaggacgagcagaaaaacaggatgatggagaccaaggaattattcgactgggtcctgaaacaaccctgttttgagaaaacatccttcatgctgttcttgaacaagttcgacatatttgagaagaaagttcttgacgttccgttgaacgtttgcgagtggttcagagattaccaaccagtttcaagtgggaaacaagagattgagcatgcatacgagtttgtgaagaagaagtttgaggagttatattaccagaacacggcgccggatagagtggacagggtattcaaaatctacaggacgacggctttggaccagaagcttgtaaagaaaacgttcaagctcgtagatgagacactaagaaggagaaatttactggaggctggccttttatga

SEQ ID NO: 24 - protein, 709 aa, Artificial Sequence
  Synthetic Construct
MSAVLTAGGGLTAGDRSIITAINTGASSLSFVGSAFIVLCYCLFKELRKFSFKLVFYLALSDMLCSFFLIVGDPSKGFICYAQGYTTHFFCVASFLWTTTIAFTLHRTVVKHKTDVEDLEAMFHLYVWGTSLVVTVIRSFGNNHSHLGPWCWTQTGLKGKAVHFLTFYAPLWGAILYNGFTYFQVIRMLRNARRMAVGMSDRVDQFDNRAELKVLNRWGYYPLILIGSWAFGTINRIHDFIEPGHKIFWLSVLDVGTAALMGLFNSIAYGFNSSVRRAIHERLELFLPERLYRWLPSNFRPKNHLILHQQQQQRSEMVSLKTEDQQMGLLCSRSRHHTEDTDENTQAAEIERRIEQEAKAEKHIRKLLLLGAGESGKSTIFKQIKLLFQTGFDEGELKSYVPVIHANVYQTIKLLHDGTKEFAQNETDSAKYMLSSESIAIGEKLSEIGGRLDYPRLTKDIAEGIETLWKDPAIQETCARGNELQVPDCTKYLMENLKRLSDINYIPTKEDVLYARVRTTGVVEIQFSPVGENKKSGEVYRLFDVGGQRNERRKWIHLFEGVTAVIFCAAISEYDQTLFEDEQKNRMMETKELFDWVLKQPCFEKTSFMLFLNKFDIFEKKVLDVPLNVCEWFRDYQPVSSGKQEIEHAYEFVKKKFEELYYQNTAPDRVDRVFKIYRTTALDQKLVKKTFKLVDETLRRRNLLEAGLL

SEQ ID NO: 25 - DNA, 22 bp, Arabidopsis thaliana
gacgccgtcatttgctactctc

SEQ ID NO: 26 - DNA, 22 bp, Arabidopsis thaliana
cgtcgtcatcctcaccttcgtt

SEQ ID NO: 27 - DNA, 18 bp, Arabidopsis thaliana
gatgagcagatctcgcag

SEQ ID NO: 28 - DNA, 21 bp, Arabidopsis thaliana
tccttgaccaaatgtatatcc

SEQ ID NO: 29 - DNA, 20 bp, Arabidopsis thaliana
cacggcgagggtaactggaa

SEQ ID NO: 30 - DNA, 20 bp, Arabidopsis thaliana
tgaagggagctccagcttca